(19) United States
(12) Patent Application Publication
(10) Pub. No.: US 2006/0073525 A1
(43) Pub. Date: Apr. 6, 2006

(54) Methods for detecting breast and ovarian cancer

(75) Inventors: George Yousef, St. John's (CA); Eleftherios P. Diamandis, Toronto (CA)

Correspondence Address: MERCHANT & GOULD PC, P.O. BOX 2903, MINNEAPOLIS, MN, 55402-0903, US

(73) Assignee: Mount Sinai Hospital, Toronto (CA)

(21) Appl. No.: 10/526,029

(22) PCT Filed: Aug. 28, 2003
(86) PCT No.: PCT/CA03/01310

§ 371 Date: Sep. 20, 2005

Related U.S. Application Data
(60) Provisional application No. 60/407,333, filed on Aug. 28, 2002.

(51) Int. Cl. G01N 33/574

(52) U.S. Cl. 435/7.23

(57) ABSTRACT

Kallikrein 5 constitutes a novel biomarker for diagnosis, treatment and monitoring of breast and ovarian cancer. A method is provided for diagnosing and monitoring breast or ovarian cancer or a predisposition to breast or ovarian cancer, in a subject comprising detecting kallikrein 5 in a sample from the subject. Imaging methods, screening methods for test agents for inhibiting breast or ovarian cancer, and therapeutic applications based on kallikrein 5 are also described.

### FIELD OF THE INVENTION

The invention relates to methods for detecting breast and ovarian cancer.

### BACKGROUND OF THE INVENTION

The human kallikrein gene family was, until recently, thought to consist of only 3 genes: pancreatic/renal kallikrein (KLK1, encoding for hK1 protein), human glandular kallikrein 2 (KLK2, encoding for hK2 protein) and human kallikrein 3 (KLK3, encoding for hK3 protein or prostate specific antigen, PSA). This gene family has already contributed two excellent biomarkers (PSA and hK2) which are currently used for diagnosis and monitoring of prostate cancer (1). More recently, new members of the human kallikrein gene family have been discovered. This gene family now includes 15 genes encoding serine proteases that show significant homologies at both the DNA and the amino acid level, and are localized in tandem on human chromosome 19q13.4. Recent developments on this gene family have been reviewed (2, 3).

The human kallikrein gene 5 (KLK5, according to the official kallikrein gene nomenclature) (4), previously known as the kallikrein-like gene-2 (KLK-L2) (5) or human stratum corneum tryptic enzyme (HSCTE) (6) is a newly identified member of the human kallikrein gene family, which maps to chromosome 19q13.4, close to other kallikrein genes (7). By RT-PCR analysis, it has been reported that this gene is mainly expressed in the skin, testis, breast and brain (5, 6). The protein sequence has the conserved catalytic triad of serine proteases (5) and the enzyme was found to have proteolytic activity (6). KLK5 gene expression at the mRNA level was also found to be regulated by steroid hormones in the BT-474 breast cancer cell line (5).

The citation of any reference herein is not an admission that such reference is available as prior art to the instant invention.

### SUMMARY OF THE INVENTION

The expression of kallikrein 5, was determined at the protein level, in various tissues and biological fluids and it was found that kallikrein 5 protein is elevated in serum of patients with ovarian and breast cancer and in the ascites fluid of patients with advanced ovarian cancer.

Therefore, kallikrein 5 has particular application in the detection of breast and ovarian cancer. Thus, kallikrein 5 constitutes a new biomarker for diagnosis and monitoring (i.e. monitoring progression or therapeutic treatment) of breast and ovarian cancer. In accordance with an aspect of the invention kallikrein 5 is used for the diagnosis, monitoring, progression, treatment, and prognosis of breast and ovarian cancer, and it may be used as a biomarker before surgery or after relapse. In another aspect of the invention, kallikrein 5 is used for the diagnosis, monitoring, and prognosis of breast and ovarian tumors. Kallikrein 5 may also be a biomarker for skin and endometrial cancer.

Kallikrein 5, and agents that bind to kallikrein 5, may be used to detect breast and ovarian cancer and they can be used in the diagnostic evaluation of breast and ovarian cancer, and the identification of subjects with a predisposition to such disorders. Methods for detecting kallikrein 5 can be used to monitor breast and ovarian cancer.

The presence of kallikrein 5 in a sample can be assessed, for example, by detecting the presence in the sample of (a) kallikrein 5 protein or fragments thereof; or (b) metabolites which are produced directly or indirectly by a kallikrein 5 protein.

In an embodiment, the invention provides a method for detecting a kallikrein 5 protein comprising (a) obtaining a sample from a patient; (b) detecting or identifying in the sample a kallikrein 5 protein; and (c) comparing the detected amount with an amount detected for a standard.

In an aspect of the invention, a method for screening a subject for breast or ovarian cancer is provided comprising (a) obtaining a biological sample from a subject; (b) detecting the amount of kallikrein 5 in said sample; and (c) comparing said amount of kallikrein 5 detected to a predetermined standard, where detection of a level of kallikrein 5 greater than that of a standard indicates disease.

The term “detect” or “detecting” includes assaying, imaging or otherwise establishing the presence or absence of the target kallikrein 5, subunits thereof, or combinations of reagent bound targets, and the like, or assaying for, imaging, ascertaining, establishing, or otherwise determining one or more factual characteristics of breast or ovarian cancer, metastasis, stage, or similar conditions. The term encompasses diagnostic, prognostic, and monitoring applications for kallikrein 5.

According to a method involving a kallikrein 5 protein the levels in a sample from a patient are compared with the normal levels of kallikrein 5 protein in samples of the same type obtained from controls (e.g. samples from individuals not afflicted with disease). Significantly altered levels in the sample of the kallikrein 5 protein relative to the normal levels in a control is indicative of disease. Thus, a method is provided of assessing whether a patient is afflicted with or has a pre-disposition to breast or ovarian cancer, comprising comparing (a) levels of kallikrein 5 in a sample from the patient; and (b) normal levels of kallikrein 5 in samples of the same type obtained from control patients not afflicted with breast or ovarian cancer, wherein significantly altered levels of kallikrein 5 relative to the corresponding normal levels of kallikrein 5, is an indication that the patient is afflicted with or has a pre-disposition to breast or ovarian cancer.

A significant difference between the levels of kallikrein 5 in the patient and the normal levels (e.g. higher levels in the patient) is an indication that the patient is afflicted with or has a predisposition to breast or ovarian cancer.

The invention further relates to a method of assessing the efficacy of a therapy for inhibiting breast or ovarian cancer in a patient. A method of the invention comprises comparing: (a) levels of kallikrein 5 in a sample from the patient obtained from the patient prior to providing at least a portion of the therapy to the patient; and (b) levels of kallikrein 5 in a second sample obtained from the patient following therapy.

A significant difference between the levels of kallikrein 5 in the second sample relative to the first sample (e.g. lower levels of kallikrein 5) is an indication that the therapy is efficacious for inhibiting breast or ovarian cancer.

The “therapy” may be any therapy for treating breast or ovarian cancer including but not limited to therapeutics, radiation, immunotherapy, gene therapy, and surgical removal of tissue. Therefore, the method can be used to evaluate a patient before, during, and after therapy.

In an aspect, the invention provides a method for monitoring the progression of breast or ovarian cancer in a patient the method comprising:
(a) detecting kallikrein 5 in a sample from the patient at a first time point;
(b) repeating step (a) at a subsequent point in time; and
(c) comparing the levels detected in (a) and (b), and therefrom monitoring the progression of the breast or ovarian cancer.

The invention also provides a method for assessing the potential efficacy of a test agent for inhibiting breast or ovarian cancer, and a method of selecting an agent for inhibiting breast or ovarian cancer.

The invention also contemplates a method of assessing the potential of a test compound to contribute to breast or ovarian cancer comprising:
(a) maintaining separate aliquots of breast or ovarian cancer diseased cells in the presence and absence of the test compound; and
(b) comparing the level of kallikrein 5 in each of the aliquots.

A significant difference between the levels of kallikrein 5 in the aliquot maintained in the presence of (or exposed to) the test compound relative to the aliquot maintained in the absence of the test compound, indicates that the test compound potentially contributes to breast or ovarian cancer.

The present invention also relates to a method for diagnosing and monitoring breast or ovarian carcinoma in a subject comprising detecting or measuring kallikrein 5 in a sample from the subject using a reagent that detects kallikrein 5, in particular antibodies specifically reactive with kallikrein 5 or a part thereof In an embodiment, the sample is serum.

In an embodiment, the invention relates to a method for diagnosing and monitoring breast or ovarian cancer in a subject by quantitating kallikrein 5 in a biological sample from the subject comprising (a) reacting the biological sample with an antibody specific for kallikrein 5 which is directly or indirectly labelled with a detectable substance; and (b) detecting the detectable substance.

In another aspect the invention provides a method for using an antibody to detect expression of a kallikrein 5 protein in a sample, the method comprising: (a) combining an antibody specific for kallikrein 5 with a sample under conditions which allow the formation of antibody:protein complexes; and (b) detecting complex formation, wherein complex formation indicates expression of the protein in the sample. Expression may be compared with standards and is diagnostic of endocrine cancer.

Embodiments of the methods of the invention involve (a) reacting a biological sample from a subject with an antibody specific for kallikrein 5 which is directly or indirectly labelled with an enzyme; (b) adding a substrate for the enzyme wherein the substrate is selected so that the substrate, or a reaction product of the enzyme and substrate forms fluorescent complexes; (c) quantitating kallikrein 5 in the sample by measuring fluorescence of the fluorescent complexes; and (d) comparing the quantitated levels to levels obtained for other samples from the subject patient, or control subjects. In an embodiment the quantitated levels are compared to levels quantitated for control subjects without ovarian cancer wherein an increase in kallikrein 5 levels compared with the control subjects is indicative of ovarian cancer. In another embodiment the quantitated levels are compared to levels quantitated for control subjects without breast cancer wherein an increase in kallikrein 5 levels compared with the control subjects is indicative of breast cancer.

A particular embodiment of the invention comprises the following steps
(a) incubating a biological sample with a first antibody specific for kallikrein 5 which is directly or indirectly labeled with a detectable substance, and a second antibody specific for kallikrein 5 which is immobilized;
(b) detecting the detectable substance thereby quantitating kallikrein 5 in the biological sample; and
(c) comparing the quantitated kallikrein 5 with levels for a predetermined standard.

The standard may correspond to levels quantitated for samples from control subjects without breast or ovarian cancer, with a different disease stage, or from other samples of the subject. Increased levels of kallikrein 5 as compared to the standard is indicative of cancer. In an embodiment, the levels are 2×, 5×, 10×, 15×, or 20× the levels of the standard.

The invention also contemplates the methods described herein using multiple markers for breast or ovarian cancer. Therefore, the invention contemplates a method for anaylzing a biological sample for the presence of kallikrein 5 and other markers that are specific indicators of breast or ovarian cancer. Other markers include human stratum corneum chymotryptic enzyme (HSCCE), haptoglobin alpha, osteopontin, kallikrein 2, kallikrein 3, kallikrein 4, kallikrein 6, kallikrein 7, kallikrein 8, kallikrein 9, kallikrein 10, kallikrein 11, Kallikrein 13, kallikrein 14, and kallikrein 15; CA125, CA15-3, CA19-9, OVX1, lysophosphatidic acid (LPA), creatin-kinase BB, and carcinoembryonic antigen (CEA). Preferably the other markers are markers to kallikreins. In an aspect of the invention, the markers are one or more of hK6, hK10, and hK11. The methods described herein may be modified by including reagents to detect the markers, or nucleic acids for the markers.

In accordance with an aspect of the invention a method is provided comprising administering to a subject an agent that has been constructed to target one or more kallikreins.

The invention also contemplates a method comprising administering to cells or tissues imaging agents that carry labels for imaging and bind to kallikrein 5 and optionally other markers of breast or ovarian cancer, and then imaging the cells or tissues.

The invention further contemplates an in vivo method comprising administering to a mammal one or more agent that carries a label for imaging and binds to kallikrein 5 and optionally other markers of breast or ovarian cancer, and then imaging the mammal.

According to a particular aspect of the invention, an in vivo method for imaging breast or ovarian cancer is provided comprising:
(a) injecting a patient with an imaging agent that binds to kallikrein 5, the imaging agent carrying a label for imaging the breast or ovarian cancer;
(b) allowing the imaging agent to incubate in vivo and bind to kallikrein 5 associated with the breast or ovarian cancer; and
(c) detecting the presence of the label localized to the breast or ovarian cancer.

In an embodiment of the invention the imaging agent is an antibody which recognizes kallikrein 5. In another embodiment of the invention the agent is a chemical entity which recognizes kallikrein 5.

The imaging agent carries a label to image kallikrein 5 and optionally other markers. Examples of labels useful for imaging are radiolabels, fluorescent labels (e.g fluorescein and rhodamine), nuclear magnetic resonance active labels, positron emitting isotopes detectable by a positron emission tomography (“PET”) scanner, chemiluminescers such as luciferin, and enzymatic markers such as peroxidase or phosphatase. Short-range radiation emitters, such as isotopes detectable by short-range detector probes can also be employed.

The invention also contemplates the localization or imaging methods described herein using multiple markers for breast or ovarian cancer. For example, an imaging method may further comprise injecting the patient with one or more of an agent that binds to human stratum corneum chymotryptic enzyme (HSCCE), haptoglobin alpha, osteopontin, kallikrein 2, kallikrein 3, kallikrein 4, kallikrein 6, kallikrein 7, kallikrein 8, kallikrein 9, kallikrein 10, kallikrein 11, kallikrein 13, kallikrein 14, kallikrein 15, CA125, CA15-3, CA19-9, OVX1, lysophosphatidic acid (LPA) or carcinoembryonic antigen (CEA).

The invention also relates to kits for carrying out the methods of the invention. In an embodiment, the kit is for assessing whether a patient is afflicted with breast or ovarian cancer, and it comprises reagents for assessing kallikrein 5.

In another aspect, the invention relates to a kit for assessing the suitability of each of a plurality of test compounds for inhibiting breast or ovarian cancer in a patient. The kit comprises reagents for assessing kallikrein 5 and optionally a plurality of test agents or compounds.

The invention contemplates a kit for assessing the presence of breast or ovarian cancer cells, wherein the kit comprises antibodies specific for kallikrein 5, and optionally antibodies specific for other markers associated with breast or ovarian cancer.

Additionally the invention provides a kit for assessing the potential of a test compound to contribute to breast or ovarian cancer. The kit comprises breast or ovarian cancer cells and reagents for assessing kallikrein 5, and optionally other markers associated with breast or ovarian cancer.

The invention also provides a diagnostic composition comprising kallikrein 5 or agents that bind to kallikrein 5 or parts thereof. Agents can be labelled with detectable substances.

The invention also relates to therapeutic applications for breast or ovarian cancer.

In an aspect, the invention relates to compositions comprising a kallikrein 5 or part thereof, or an antibody specific for kallikrein 5, and a pharmaceutically acceptable carrier, excipient, or diluent. A method for treating or preventing breast or ovarian cancer in a patient is also provided comprising administering to a patient in need thereof, a kallikrein 5 protein or part thereof, an antibody specific for kallikrein 5, or a composition of the invention. In an aspect the invention provides a method of treating a patient afflicted with or at risk of developing breast or ovarian cancer comprising inhibiting expression of kallikrein 5.

In an aspect, the invention provides antibodies specific for kallikrein 5 that can be used therapeutically to destroy or inhibit the growth of kallikrein 5 expressing cancer cells, (e.g. ovarian cancer or breast cancer cells), or to block kallikrein 5 activity. In addition, kallikrein 5 proteins may be used in various immunotherapeutic methods to promote immune-mediated destruction or growth inhibition of tumors expressing kallikrein 5.

The invention also contemplates a method of using kallikrein 5 or part thereof, or an antibody specific for kallikrein 5 in the preparation or manufacture of a medicament for the prevention or treatment of breast or ovarian cancer.

Another aspect of the invention is the use of kallikrein 5, peptides derived therefrom, or chemically produced (synthetic) peptides, or any combination of these molecules, for use in the preparation of vaccines to prevent breast or ovarian cancer and/or to treat breast or ovarian cancer.

The invention contemplates vaccines for stimulating or enhancing in a subject to whom the vaccine is administered production of antibodies directed against kallikrein 5.

The invention also provides a method for stimulating or enhancing in a subject production of antibodies directed against kallikrein 5. The method comprises administering to the subject a vaccine of the invention in a dose effective for stimulating or enhancing production of the antibodies.

The invention further provides a method for treating, preventing, or delaying recurrence of breast or ovarian cancer. The methods comprise administering to the subject a vaccine of the invention in a dose effective for treating, preventing, or delaying recurrence of breast or ovarian cancer.

The invention also contemplates the methods, compositions, and kits described herein using additional markers associated with breast or ovarian cancer. The methods described herein may be modified by including reagents to detect the additional markers, or nucleic acids for the markers.

Other objects, features and advantages of the present invention will become apparent from the following detailed description. It should be understood, however, that the detailed description and the specific examples while indicating preferred embodiments of the invention are given by way of illustration only, since various changes and modifications within the spirit and scope of the invention will become apparent to those skilled in the art from this detailed description.

### DESCRIPTION OF THE DRAWINGS

The invention will now be described in relation to the drawings in which:

FIG. 1 is a calibration curve for the hK5 immunofluorometric ELISA assay. The detection limit is 0.1 μg/L and the dynamic range extends to 25 μg/L.

FIG. 2 shows the hK5 content of cytosolic extracts of various adult and fetal human tissues. All hK5 concentrations were corrected for the amount of total protein.

FIG. 3 shows the hK5 protein content (ng/mg of total protein) of cytosolic extracts of normal ovaries, benign ovarian tissue and ovarian cancer. Note the higher levels of hK5 in about 55-60% of extracts from benign ovarian tissue and cancer. Highest levels are seen in cancer tissues. N=number of tissues extracted. The horizontal line indicates 100^{th} percentile for normal tissues.

FIG. 4 shows the distribution of hK5 concentration in serum of patients diagnosed with various malignancies. Sera from normal female and male subjects were also included. High hK5 concentration (>100^{th} percentile of normals) is found in a proportion of patients with ovarian and breast cancers.

FIG. 5 shows the results of the fractionation of three biological fluids (serum, ascites fluid from an ovarian cancer patient and breast milk) by size-exclusion liquid chromatography. The elution profile of molecular weight standards is denoted by arrows. In serum, hK5 elutes as two immumoreactive peaks, one with an apparent molecular weight of 50 kDa (fractions 37-39) and another one, with an apparent molecular weight of approximately 150-180 kDa (fractions 31-33). The elution profile of another kallikrein with a similar theoretical molecular weight, hK6, is also shown with broken lines. This kallikrein elutes at the molecular weight of about 35 kDa, corresponding to free hK6. In ascites fluid, the same comments apply as for serum. In breast milk, hK5 elutes mainly as a single immunoreactive peak. hK6, elutes as two distinct peaks, one at the molecular weight of about 35 kDa and another one, at a molecular weight of about 100 kDa. These data confirm that at least in serum and ascites fluid, hK5 likely interacts with serum proteinase inhibitors or other interacting proteins.

FIG. 6 shows the expression of hK5 protein by various cell lines. On the Y axis, hK5 concentration in tissue culture supernatants is shown after incubation with steroids, at a concentration of 10⁻⁸ M, for six days. Ethanol is used as control. Note the up-regulation of hK5 expression by norgestrel and DHT in the PC-3 (AR₆) prostate cancer cell line, the up-regulation of hK5 expression by estradiol in the MCF-7 breast carcinoma cell line and the down-regulation of hK5 expression by dexamethasone in the HTB-75 ovarian cancer cell line.

### DETAILED DESCRIPTION OF THE INVENTION

The invention relates to newly discovered correlations between expression of kallikrein 5 and breast and ovarian cancer. The methods described herein provide sensitive methods for detecting breast or ovarian cancer. The level of expression of kallikrein 5 correlates with the presence of breast or ovarian cancer. Methods are provided for detecting the presence of breast or ovarian cancer in a sample, the absence of breast or ovarian cancer in a sample, assessing the histology of tissues associated with breast or ovarian cancer, and other characteristics of breast or ovarian cancer that are relevant to prevention, diagnosis, characterization, and therapy of breast or ovarian cancer in a patient. Methods are also provided for assessing the efficacy of one or more test agents for inhibiting kallikrein 5 that affect breast or ovarian cancer, assessing the efficacy of a therapy for breast or ovarian cancer, monitoring the progression of breast or ovarian cancer, selecting an agent or therapy for inhibiting breast or ovarian cancer, treating a patient afflicted with breast or ovarian cancer, inhibiting breast or ovarian cancer in a patient, and assessing the potential of a test compound to contribute to breast or ovarian cancer.

Glossary

Samples that may be analyzed using the methods of the invention include those which are known or suspected to express kallikrein 5 or contain kallikrein 5. The terms “sample”, “biological sample”, and the like mean a material known or suspected of expressing or containing kallikrein 5, in particular kallikrein 5 associated with breast or ovarian cancer. The test sample can be used directly as obtained from the source or following a pretreatment to modify the character of the sample. The sample can be derived from any biological source, such as tissues, extracts, or cell cultures, including cells (e.g. tumor cells), cell lysates, and physiological fluids, such as, for example, whole blood, plasma, serum, saliva, ocular lens fluid, cerebral spinal fluid, sweat, urine, milk, ascites fluid, synovial fluid, peritoneal fluid and the like. In an embodiment, the sample is serum. The sample can be obtained from animals, preferably mammals, most preferably humans. The sample can be treated prior to use, such as preparing plasma from blood, diluting viscous fluids, and the like. Methods of treatment can involve filtration, distillation, extraction, concentration, inactivation of interfering components, the addition of reagents, and the like. Proteins may be isolated from the samples and utilized in the methods of the invention.

The terms “subject”, “individual” or “patient” refer to a warm-blooded animal such as a mammal, which is suspected of having or being pre-disposed to breast or ovarian cancer or condition as described herein. In particular, the terms refer to a human.

The term “kallikrein 5” or “kallikrein 5 protein” includes human kallikrein 5 (“hK5”), in particular the native-sequence polypeptide, isoforms, chimeric polypeptides, all homologs, fragments, and precursors of human kallikrein 5. The amino acid sequence for native hK5 include the sequences of GenBank Accession Nos. AF135028 and AAD26429 and shown in SEQ ID NO. 1.

A “native-sequence polypeptide” comprises a polypeptide having the same amino acid sequence of a polypeptide derived from nature. Such native-sequence polypeptides can be isolated from nature or can be produced by recombinant or synthetic means. The term specifically encompasses naturally occurring truncated or secreted forms of a polypeptide, polypeptide variants including naturally occurring variant forms (e.g. alternatively spliced forms or splice variants), and naturally occurring allelic variants.

The term “polypeptide variant” means a polypeptide having at least about 70-80%, preferably at least about 85%, more preferably at least about 90%, most preferably at least about 95% amino acid sequence identity with a native-sequence polypeptide, in particular having at least 70-80%, 85%, 90%, 95% amino acid sequence identity to the sequences identified in the GenBank Accession Nos. AF135028 and AAD26429, and shown in SEQ ID NO. 1. Such variants include, for instance, polypeptides wherein one or more amino acid residues are added to, or deleted from, the N- or C-terminus of the full-length or mature sequences of SEQ ID NO: 1, including variants from other species, but excludes a native-sequence polypeptide.

An allelic variant may also be created by introducing substitutions, additions, or deletions into a nucleic acid encoding a native polypeptide sequence such that one or more amino acid substitutions, additions, or deletions are introduced into the encoded protein. Mutations may be introduced by standard methods, such as site-directed mutagenesis and PCR-mediated mutagenesis. In an embodiment, conservative substitutions are made at one or more predicted non-essential amino acid residues. A “conservative amino acid substitution” is one in which an amino acid residue is replaced with an amino acid residue with a similar side chain. Amino acids with similar side chains are known in the art and include amino acids with basic side chains (e.g. Lys, Arg, His), acidic side chains (e.g. Asp, Glu), uncharged polar side chains (e.g. Gly, Asp, Glu, Ser, Thr, Tyr and Cys), nonpolar side chains (e.g. Ala, Val, Leu, Iso, Pro, Trp), beta-branched side chains (e.g. Thr, Val, Iso), and aromatic side chains (e.g. Tyr, Phe, Trp, His). Mutations can also be introduced randomly along part or all of the native sequence, for example, by saturation mutagenesis. Following mutagenesis the variant polypeptide can be recombinantly expressed and the activity of the polypeptide may be determined.

Polypeptide variants include polypeptides comprising amino acid sequences sufficiently identical to or derived from the amino acid sequence of a native polypeptide which include fewer amino acids than the full length polypeptides. A portion of a polypeptide can be a polypeptide which is for example, 10, 15, 20, 25, 30, 35, 40, 45, 50, 60, 70, 80, 90, 100 or more amino acids in length. Portions in which regions of a polypeptide are deleted can be prepared by recombinant techniques and can be evaluated for one or more functional activities such as the ability to form antibodies specific for a polypeptide.

A naturally occurring allelic variant may contain conservative amino acid substitutions from the native polypeptide sequence or it may contain a substitution of an amino acid from a corresponding position in a kallikrein polypeptide homolog, for example, the murine kallikrein polypeptide.

The invention also includes polypeptides that are substantially identical to the sequences of GenBank Accession Nos. AF135028 and AAD26429 and shown in SEQ ID NO. 1 (e.g. at least about 45%, preferably 50%, 55%, 60%, 65%, 70%, 75%, 80%, 85%, 90%, 95%, 97%, 98%, or 99%, sequence identity), and in particular polypeptides that retain the immunogenic activity of the corresponding native-sequence polypeptide.

Percent identity of two amino acid sequences, or of two nucleic acid sequences identified herein is defined as the percentage of amino acid residues or nucleotides in a candidate sequence that are identical with the amino acid residues in a polypeptide or nucleic acid sequence, after aligning the sequences and introducing gaps, if necessary, to achieve the maximum percent sequence identity, and not considering any conservative substitutions as part of the sequence identity. Alignment for purposes of determining percent amino acid or nucleic acid sequence identity can be achieved in various conventional ways, for instance, using publicly available computer software including the GCG program package (Devereux J. et al., Nucleic Acids Research 12(1): 387, 1984); BLASTP, BLASTN, and FASTA (Atschul, S. F. et al. J. Molec. Biol. 215: 403-410, 1990). The BLAST X program is publicly available from NCBI and other sources (BLAST Manual, Altschul, S. et al. NCBI NLM NIH Bethesda, Md. 20894; Altschul, S. et al. J. Mol. Biol. 215: 403-410, 1990). Skilled artisans can determine appropriate parameters for measuring alignment, including any algorithms needed to achieve maximal alignment over the full length of the sequences being compared. Methods to determine identity and similarity are codified in publicly available computer programs.

Kallikrein 5 polypeptides include chimeric or fusion proteins. A “chimeric protein” or “fusion protein” comprises all or part (preferably biologically active) of a kallikrein 5 polypeptide operably linked to a heterologous polypeptide (i.e., a polypeptide other than a kallikrein 5 polypeptide). Within the fusion protein, the term “operably linked” is intended to indicate that a kallikrein 5 polypeptide and the heterologous polypeptide are fused in-frame to each other. The heterologous polypeptide can be fused to the N-terminus or C-terminus of a kallikrein 5 polypeptide. A useful fusion protein is a GST fusion protein in which a kallikrein 5 polypeptide is fused to the C-terminus of GST sequences. Another example of a fusion protein is an immunoglobulin fusion protein in which all or part of a kallikrein 5 polypeptide is fused to sequences derived from a member of the immunoglobulin protein family. Chimeric and fusion proteins can be produced by standard recombinant DNA techniques.

Kallikrein polypeptides may be isolated from a variety of sources, such as from human tissue types or from another source, or prepared by recombinant or synthetic methods, or by any combination of these and similar techniques.

The terms “KLK5” or “KLK5 nucleic acid(s)” are intended to include DNA and RNA (e.g. mRNA) and can be either double stranded or single stranded. The terms include but are not limited to nucleic acids that encode a native-sequence polypeptide, a polypeptide variant including a portion of a kallikrein polypeptide, an isoform, precursor, and chimeric polypeptide. The nucleic acid sequences encoding native kallikrein polypeptides employed in the present invention include the nucleic acid sequences of GenBank Accession No. AF135028 and in SEQ ID NO: 2, or fragments thereof.

“Statistically different levels” or “significant difference” in levels of markers in a patient sample compared to a control or standard (e.g. normal levels or levels in other samples from a patient) may represent levels that are higher or lower, in particular higher, than the standard error of the detection assay. The levels may be at least 1.5, 2, 3, 4, 5, or 6 times higher or lower, in particular higher, than the control or standard. In particular methods of the invention the levels of kallikrein 5 are about 2×, 5×, 10×, 15×, or 20× the levels of the standard.

“Binding agent” refers to a substance such as a polypeptide or antibody that specifically binds to a kallikrein 5. A substance “specifically binds” to a kallikrein 5 if it reacts at a detectable level with a kallikrein 5, and does not react detectably with peptides containing unrelated sequences or sequences of different polypeptides. Binding properties may be assessed using an ELISA, which may be readily performed by those skilled in the art (see for example, Newton et al, Develop. Dynamics 197: 1-13, 1993).

A binding agent may be a ribosome, with or without a peptide component, an RNA molecule, or a polypeptide. A binding agent may be a polypeptide that comprises a kallikrein 5 polypeptide sequence, a peptide variant thereof, or a non-peptide mimetic of such a sequence. By way of example a kallikrein 5 sequence may be a peptide portion of a kallikrein 5 that is capable of modulating a function mediated by the kallikrein 5.

Methods for Detecting Kallikrein 5

A variety of methods can be employed for the diagnostic and prognostic evaluation of breast and ovarian cancer involving kallikrein 5, and the identification of subjects with a predisposition to such disorders. Such methods may, for example, utilize binding agents (e.g. antibodies) directed against kallikrein 5, including peptide fragments. In particular, antibodies may be used, for example, for the detection of either an over- or an under-abundance of kallikrein 5 relative to a non-disorder state or the presence of a modified (e.g., less than full length) kallikrein 5 which correlates with a disorder state, or a progression toward a disorder state.

The invention also contemplates a method for detecting breast or ovarian cancer comprising producing a profile of levels of kallikrein 5 and other markers associated with breast or ovarian cancer in cells from a patient, and comparing the profile with a reference to identify a profile for the test cells indicative of disease.

The methods described herein may be used to evaluate the probability of the presence of malignant or pre-malignant cells, for example, in a group of cells freshly removed from a host. Such methods can be used to detect tumors, quantitate their growth, and help in the diagnosis and prognosis of disease. For example, higher levels of kallikrein 5 may be indicative of advanced disease, e.g. advanced ovarian cancer. The methods can be used to detect the presence of cancer metastasis, as well as confirm the absence or removal of all tumor tissue following surgery, cancer chemotherapy, and/or radiation therapy. They can further be used to monitor cancer chemotherapy and tumor reappearance.

The methods described herein can be adapted for diagnosing and monitoring breast and ovarian carcinoma by detecting kallikrein 5 in biological samples from a subject. These applications require that the amount of kallikrein 5 quantitated in a sample from a subject being tested be compared to levels quantitated for another sample or an earlier sample from the subject, or levels quantitated for a control sample. Levels for control samples from healthy subjects or breast or ovarian cancer subjects may be established by prospective and/or retrospective statistical studies. Healthy subjects who have no clinically evident disease or abnormalities may be selected for statistical studies. Diagnosis may be made by a finding of statistically different levels of kallikrein 5 compared to a control sample or previous levels quantitated for the same subject.

Binding agents specific for kallikrein 5 may be used for a variety of diagnostic and assay applications. There are a variety of assay formats known to the skilled artisan for using a binding agent to detect a target molecule in a sample. (For example, see Harlow and Lane, Antibodies: A Laboratory Manual, Cold Spring Harbor Laboratory, 1988). In general, the presence or absence of breast or ovarian cancer in a subject may be determined by (a) contacting a sample from the subject with a binding agent for kallikrein 5; (b) detecting in the sample levels of kallikrein 5 that bind to the binding agent; and (c) comparing the levels of kallikrein 5 with a predetermined standard or cut-off value.

In particular embodiments, the binding agent is an antibody.

In an aspect, the invention provides a diagnostic method for monitoring or diagnosing breast or ovarian cancer in a subject by quantitating kallikrein 5 in a biological sample from the subject comprising reacting the sample with antibodies specific for kallikrein 5, which are directly or indirectly labeled with a detectable substance and detecting the detectable substance.

In an aspect of the invention, a method for detecting breast or ovarian cancer is provided comprising:
(a) obtaining a sample suspected of containing kallikrein 5 associated with breast or ovarian cancer;
(b) contacting said sample with antibodies that specifically bind kallikrein 5 under conditions effective to bind the antibodies and form complexes;
(c) measuring the amount of kallikrein 5 present in the sample by quantitating the amount of the complexes; and
(d) comparing the amount of kallikrein 5 present in the samples with the amount of kallikrein 5 in a control, wherein a change or significant difference in the amount of kallikrein 5 in the sample compared with the amount in the control is indicative of breast or ovarian cancer.

In an embodiment, the invention contemplates a method for monitoring the progression of breast or ovarian cancer in an individual, comprising:
(a) contacting an amount of an antibody which binds to a kallikrein 5 protein, with a sample from the individual so as to form a complex comprising the antibody and kallikrein 5 protein in the sample;
(b) determining or detecting the presence or amount of complex formation in the sample;
(c) repeating steps (a) and (b) at a point later in time; and
(d) comparing the result of step (b) with the result of step (c), wherein a difference in the amount of complex formation is indicative of disease, disease stage, and/or progression of the cancer in said individual.

In an embodiment, the invention contemplates a method for monitoring the progression of breast or ovarian cancer in an individual, comprising:
(a) contacting antibodies which bind to kallikrein 5 with a sample from the individual so as to form binary complexes comprising the antibodies and kallikrein 5 in the sample;
(b) determining or detecting the presence or amount of complex formation in the sample;
(c) repeating steps (a) and (b) at a point later in time; and
(d) comparing the result of step (b) with the result of step (c), wherein a difference in the amount of complex formation is indicative of the stage and/or progression of the breast or ovarian cancer in said individual.

The amount of complexes may also be compared to a value representative of the amount of the complexes from an individual not at risk of, or afflicted with, breast or ovarian cancer at different stages. An increase in complex formation may be indicative of advanced disease e.g. advanced ovarian cancer, or an unfavourable prognosis.

Thus, antibodies specifically reactive with a kallikrein 5 protein, or derivatives, such as enzyme conjugates or labeled derivatives, may be used to detect kallikrein 5 protein in various samples (e.g. biological materials). They may be used as diagnostic or prognostic reagents and they may be used to detect abnormalities in the level of kallikrein 5 expression, or abnormalities in the structure, and/or temporal, tissue, cellular, or subcellular location of kallikrein 5. Antibodies may also be used to screen potentially therapeutic compounds in vitro to determine their effects on disorders (e.g. ovarian cancer) involving a kallikrein 5 protein, and other conditions. In vitro immunoassays may also be used to assess or monitor the efficacy of particular therapies.

Antibodies may be used in any known immunoassays that rely on the binding interaction between an antigenic determinant of a kallikrein 5 protein and the antibodies. Examples of such assays are radioimmunoassays, enzyme immunoassays (e.g. ELISA), immunofluorescence, immunoprecipitation, latex agglutination, hemagglutination, and histochemical tests. The antibodies may be used to detect and quantify kallikrein 5 in a sample in order to diagnose and treat pathological states.

In particular, the antibodies may be used in immunohistochemical analyses, for example, at the cellular and sub-subcellular level, to detect a kallikrein 5 protein, to localize it to particular breast or ovarian tumor cells and tissues, and to specific subcellular locations, and to quantitate the level of expression.

Antibodies for use in the present invention include but are not limited to monoclonal or polyclonal antibodies, immunologically active fragments (e.g. a Fab or (Fab)₂ fragments), antibody heavy chains, humanized antibodies, antibody light chains, genetically engineered single chain Fᵥ molecules (Ladner et al, U.S. Pat. No. 4,946,778), chimeric antibodies, for example, antibodies which contain the binding specificity of murine antibodies, but in which the remaining portions are of human origin, or derivatives, such as enzyme conjugates or labeled derivatives.

Antibodies including monoclonal and polyclonal antibodies, fragments and chimeras, may be prepared using methods known to those skilled in the art. Isolated native or recombinant kallikrein 5 may be utilized to prepare antibodies. See, for example, Kohler et al. (1975) Nature 256:495-497; Kozbor et al. (1985) J. Immunol Methods 81:31-42; Cote et al. (1983) Proc Natl Acad Sci 80:2026-2030; and Cole et al. (1984) Mol Cell Biol 62:109-120 for the preparation of monoclonal antibodies; Huse et al. (1989) Science 246:1275-1281 for the preparation of monoclonal Fab fragments; and, Pound (1998) Immunochemical Protocols, Humana Press, Totowa, N.J. for the preparation of phagenid or B-lymphocyte immunoglobulin libraries to identify antibodies. Antibodies specific for kallikrein 5 may also be obtained from scientific or commercial sources.

In an embodiment of the invention, antibodies are reactive against kallikrein 5 if they bind with a Kₐ of greater than or equal to 10⁻⁷ M.

In a sandwich immunoassay of the invention mouse polyclonal/monoclonal antibodies specific for kallikrein 5 and rabbit polyclonal/monoclonal antibodies specific for kallikrein 5 are utilized.

An antibody specific for kallikrein 5 may be labelled with a detectable substance and localised in biological samples based upon the presence of the detectable substance. Examples of detectable substances include, but are not limited to, the following: radioisotopes (e.g., ³H, ¹⁴C, ³⁵S, ¹²⁵I, ¹³¹I) fluorescent labels (e.g., FITC, rhodamine, lanthanide phosphors), luminescent labels such as luminol; enzymatic labels (e.g., horseradish peroxidase, beta-gal actosidase, luciferase, alkaline phosphatase, acetylcholinesterase), biotinyl groups (which can be detected by marked avidin e.g., streptavidin containing a fluorescent marker or enzymatic activity that can be detected by optical or calorimetric methods), predetermined polypeptide epitopes recognized by a secondary reporter (e.g., leucine zipper pair sequences, binding sites for secondary antibodies, metal binding domains, epitope tags). In some embodiments, labels are attached via spacer arms of various lengths to reduce potential steric hindrance. Antibodies may also be coupled to electron dense substances, such as ferritin or colloidal gold, which are readily visualised by electron microscopy.

Indirect methods may also be employed in which the primary antigen-antibody reaction is amplified by the introduction of a second antibody, having specificity for the antibody reactive against kallikrein 5. By way of example, if the antibody having specificity against kallikrein 5 is a rabbit IgG antibody, the second antibody may be goat anti-rabbit IgG, Fc fragment specific antibody labelled with a detectable substance as described herein.

Methods for conjugating or labelling the antibodies discussed above may be readily accomplished by one of ordinary skilled in the art. (See for example Inman, Methods In Enzymology, Vol. 34, Affinity Techniques, Enzyme Purification: Part B, Jakoby and Wichek (eds.), Academic Press, New York, p. 30, 1974; and Wilchek and Bayer, “The Avidin-Biotin Complex in Bioanalytical Applications,” Anal. Biochem. 171:1-32, 1988 re methods for conjugating or labelling the antibodies with enzyme or ligand binding partner).

Cytochemical techniques known in the art for localizing antigens using light and electron microscopy may be used to detect a kallikrein 5 protein. Generally, an antibody may be labeled with a detectable substance and a kallikrein 5 protein may be localised in tissues and cells based upon the presence of the detectable substance.

In the context of the methods of the invention, the sample, binding agents (e.g. antibodies) or kallikrein 5 may be immobilized on a carrier or supports Examples of suitable carriers or supports are agarose, cellulose, nitrocellulose, dexta, Sephadex, Sepharose, liposomes, carboxymethyl cellulose, polyacrylamides, polystyrene, gabbros, filter paper, magnetite, ion-exchange resin, plastic film, plastic tube, glass, polyamine-methyl vinyl-ether-maleic acid copolymer, amino acid copolymer, ethylene-maleic acid copolymer, nylon, silk, etc. The support material may have any possible configuration including spherical (e.g. bead), cylindrical (e.g. inside surface of a test tube or well, or the external surface of a rod), or flat (e.g. sheet, test strip). Thus, the carrier may be in the shape of, for example, a tube, test plate, well, beads, disc, sphere, etc. The immobilized material may be prepared by reacting the material with a suitable insoluble carrier using known chemical or physical methods, for example, cyanogen bromide coupling. Binding agents (e.g. antibodies) may be indirectly immobilized using second binding agents specific for the first binding agent. For example, mouse antibodies specific for kallikrein 5 may be immobilized using sheep anti-mouse IgG Fc fragment specific antibody coated on the carrier or support.

Where a radioactive label is used as a detectable substance, a kallikrein 5 protein may be localized by radioautography. The results of radioautography may be quantitated by determining the density of particles in the radioautographs by various optical methods, or by counting the grains.

Time-resolved fluorometry may be used to detect a fluorescent signal, label, or detectable substance. For example, the method described in Christopoulos T. K. and Diamandis E P Anal. Chem., 1992:64:342-346 may be used with a conventional time-resolved fluorometer.

Therefore, in accordance with an embodiment of the invention, a method is provided wherein a kallikrein 5 antibody is directly or indirectly labelled with an enzyme, a substrate for the enzyme is added wherein the substrate is selected so that the substrate, or a reaction product of the enzyme and substrate, forms fluorescent complexes with a lanthanide metal (e.g. europium, terbium, samarium, and dysprosium, preferably europium and terbium). A lanthanide metal is added and kallikrein 5 is quantitated in the sample by measuring fluorescence of the fluorescent complexes. Enzymes are selected based on the ability of a substrate of the enzyme, or a reaction product of the enzyme and substrate, to complex with lanthanide metals such as europium and terbium. Suitable enzymes and substrates that provide fluorescent complexes are described in U.S. Pat. No. 5,3112,922 to Diamandis. Examples of suitable enzymes include alkaline phosphatase and β-galactosidase. Preferably, the enzyme is alkaline phosphatase.

Examples of enzymes and substrates for enzymes that provide such fluorescent complexes are described in U.S. Pat. No. 5,312,922 to Diamandis. By way of example, when the antibody is directly or indirectly labelled with alkaline phosphatase the substrate employed in the method may be 4-methylumbelliferyl phosphate, 5-fluorosalicyl phosphate, or diflunisal phosphate. The fluorescence intensity of the complexes is typically measured using a time-resolved fluorometer e.g. a CyberFluor 615 Imunoanalyzer (Nordion International, Kanata, Ontario).

Antibodies specific for kallikrein 5 may also be indirectly labelled with enzymes. For example, an antibody may be conjugated to one partner of a ligand binding pair, and the enzyme may be coupled to the other partner of the ligand binding pair. Representative examples include avidin-biotin, and riboflavin-riboflavin binding protein. In an embodiment, an antibody specific for the anti-kallikrein 5 antibody is labelled with an enzyme.

In accordance with an embodiment, the present invention provides means for determining kallikrein 5 in a serum sample by measuring kallikrein 5 by immunoassay. It will be evident to a skilled artisan that a variety of immunoassay methods can be used to measure kallikrein 5 in serum. In general, a kallikrein 5 immunoassay method may be competitive or noncompetitive. Competitive methods typically employ an immobilized or immobilizable antibody to kallikrein 5 (anti-kallikrein 5) and a labeled form of kallikrein 5. Sample kallikrein 5 and labeled kallikrein 5 compete for binding to anti-kallikrein 5. After separation of the resulting labeled kallikrein 5 that has become bound to anti-kallikrein 5 (bound fraction) from that which has remained unbound (unbound fraction), the amount of the label in either bound or unbound fraction is measured and may be correlated with the amount of kallikrein 5 in the test sample in any conventional manner, e.g., by comparison to a standard curve.

In an aspect, a non-competitive method is used for the determination of kallikrein 5, with the most common method being the “sandwich” method. In this assay, two anti- kallikrein 5 antibodies are employed. One of the anti- kallikrein 5 antibodies is directly or indirectly labeled (sometimes referred to as the “detection antibody”) and the other is immobilized or immobilizable (sometimes referred to as the “capture antibody”). The capture and detection antibodies can be contacted simultaneously or sequentially with the test sample. Sequential methods can be accomplished by incubating the capture antibody with the sample, and adding the detection antibody at a predetermined time thereafter (sometimes referred to as the “forward” method); or the detection antibody can be incubated with the sample first and then the capture antibody added (sometimes referred to as the “reverse” method). After the necessary incubation(s) have occurred, to complete the assay, the capture antibody may be separated from the liquid test mixture, and the label may be measured in at least a portion of the separated capture antibody phase or the remainder of the liquid test mixture. Generally it is measured in the capture antibody phase since it comprises kallikrein 5 bound by (“sandwiched” between) the capture and detection antibodies. In another embodiment, the label may be measured without separating the capture antibody and liquid test mixture.

In a typical two-site immunometric assay for kallikrein 5, one or both of the capture and detection antibodies are polyclonal antibodies or one or both of the capture and detection antibodies are monoclonal antibodies (i.e. polyclonal/polyclonal, monoclonal/monoclonal, or monoclonal/polyclonal). The label used in the detection antibody can be selected from any of those known conventionally in the art. The label may be an enzyme or a chemiluminescent moiety, but it can also be a radioactive isotope, a fluorophor, a detectable ligand (e.g., detectable by a secondary binding by a labeled binding partner for the ligand), and the like. Preferably the antibody is labelled with an enzyme which is detected by adding a substrate that is selected so that a reaction product of the enzyme and substrate forms fluorescent complexes. The capture antibody may be selected so that it provides a means for being separated from the remainder of the test mixture. Accordingly, the capture antibody can be introduced to the assay in an already immobilized or insoluble form, or can be in an immobilizable form, that is, a form which enables immobilization to be accomplished subsequent to introduction of the capture antibody to the assay. An immobilized capture antibody may comprise an antibody covalently or noncovalently attached to a solid phase such as a magnetic particle, a latex particle, a microtiter plate well, a bead, a cuvette, or other reaction vessel. An example of an immobilizable capture antibody is antibody which has been chemically modified with a ligand moiety, e.g., a hapten, biotin, or the like, and which can be subsequently immobilized by contact with an immobilized form of a binding partner for the ligand, e.g., an antibody, avidin, or the like. In an embodiment, the capture antibody may be immobilized using a species specific antibody for the capture antibody that is bound to the solid phase.

A particular sandwich immunoassay method of the invention employs two antibodies reactive against kallikrein 5, a second antibody having specificity against an antibody reactive against kallikrein 5 labelled with an enzymatic label, and a fluorogenic substrate for the enzyme. In an embodiment, the enzyme is alkaline phosphatase (ALP) and the substrate is 5-fluorosalicyl phosphate. ALP cleaves phosphate out of the fluorogenic substrate, 5-fluorosalicyl phosphate, to produce 5-fluorosalicylic acid (FSA). 5-Fluorosalicylic acid can then form a highly fluorescent ternary complex of the form FSA-Tb(³⁺)-EDTA, which can be quantified by measuring the Tb³⁺ fluorescence in a time-resolved mode. Fluorescence intensity is measured using a time-resolved fluorometer as described herein.

The above-described immunoassay methods and formats are intended to be exemplary and are not limiting.

The invention also contemplates the methods described herein using multiple markers for breast or ovarian cancer. Therefore, the invention contemplates a method for anaylzing a biological sample for the presence of kallikrein 5 and other markers that are specific indicators of breast or ovarian cancer. Other markers include human stratum corneum chymotryptic enzyme (HSCCE), haptoglobin alpha, osteopontin, kallikrein 2, kallikrein 3, kallikrein 4, kallikrein 6, kallikrein 7, kallikrein 8, kallikrein 9, kallikrein 10, kallikrein 11, kallikrein 14, and kallikrein 15; CA125, CA15-3, CA19-9, OVX1, lysophosphatidic acid (LPA), creatin-kinase BB, and carcinoembryonic antigen (CEA). In particular aspects of the invention the other markers are markers to kallikreins. In an aspect of the invention, the markers are one or more of hK4, hK6, hK7, hK8, hK9, hK10, hK11, hK13, hK14, and hK15, more particularly hK4, hK6, hK7, hK10 and hK15, hK8, hK9, hK11, and hK14, or hK6, hK10, and hK11. The methods described herein may be modified by including reagents to detect the markers, or nucleic acids for the markers. The methods described herein may also include reagents to detect KLK5. Techniques for detecting nucleic acid such as polymerase chain reaction (PCR) and hybridization assays are well known in the art.

Computer Systems

Computer readable media comprising kallikrein 5 and optionally other markers of breast or ovarian cancer is also provided. “Computer readable media” refers to any medium that can be read and accessed directly by a computer, including but not limited to magnetic storage media, such as floppy discs, hard disc storage medium, and magnetic tape; optical storage media such as CD-ROM; electrical storage media such as RAM and ROM; and hybrids of these categories such as magnetic/optical storage media. Thus, the invention contemplates computer readable medium having recorded thereon markers identified for patients and controls.

“Recorded” refers to a process for storing information on computer readable medium. The skilled artisan can readily adopt any of the presently known methods for recording information on computer readable medium to generate manufactures comprising information on kallikrein 5 and optionally other breast and ovarian cancer markers.

A variety of data processor programs and formats can be used to store information on kallikrein 5 and other breast and ovarian cancer markers on computer readable medium. For example, the information can be represented in a word processing text file, formatted in commercially-available software such as WordPerfect and MicroSoft Word, or represented in the form of an ASCII file, stored in a database application, such as DB2, Sybase, Oracle, or the like. Any number of dataprocessor structuring formats (e.g., text file or database) may be adapted in order to obtain computer readable medium having recorded thereon the marker information.

By providing the marker information in computer readable form, one can routinely access the information for a variety of purposes. For example, one skilled in the art can use the information in computer readable form to compare marker information obtained during or following therapy with the information stored within the data storage means.

The invention provides a medium for holding instructions for performing a method for determining whether a patient has breast or ovarian cancer or a pre-disposition to breast or ovarian cancer, comprising determining the presence or absence of kallikrein 5 and optionally other breast or ovarian cancer markers, and based on the presence or absence of the kallikrein 5 and optionally other markers, determining whether the patient has breast or ovarian cancer or a pre-disposition to breast or ovarian cancer, and optionally recommending treatment for the breast or ovarian cancer or pre-disease condition.

The invention also provides in an electronic system and/or in a network, a method for determining whether a subject has breast or ovarian cancer or a pre-disposition to breast or ovarian cancer, comprising determining the presence or absence of kallikrein 5 and optionally other breast or ovarian cancer markers, and based on the presence or absence of the kallikrein 5 and optionally other markers, determining whether the subject has breast or ovarian cancer or a pre-disposition to breast or ovarian cancer, and optionally recommending treatment for the a breast or ovarian cancer or pre-disease condition.

The invention further provides in a network, a method for determining whether a subject has breast or ovarian cancer or a pre-disposition to breast or ovarian cancer comprising: (a) receiving phenotypic information on the subject and information on kallikrein 5 and optionally other breast or ovarian cancer markers associated with samples from the subject; (b) acquiring information from the network corresponding to the kallikrein 5 and optionally other markers; and (c) based on the phenotypic information and information on the kallikrein 5 and optionally other markers determining whether the subject has breast or ovarian cancer or a pre-disposition to breast or ovarian cancer; and (d) optionally recommending treatment for the breast or ovarian cancer or pre-disease condition.

The invention still further provides a system for identifying selected records that identify a breast or ovarian cancer cell or tissue. A system of the invention generally comprises a digital computer; a database server coupled to the computer; a database coupled to the database server having data stored therein, the data comprising records of data comprising kallikrein 5 and optionally other breast or ovarian cancer markers, or nucleic acids encoding same, and a code mechanism for applying queries based upon a desired selection criteria to the data file in the database to produce reports of records which match the desired selection criteria.

In an aspect of the invention a method is provided for detecting breast or ovarian cancer tissue or cells using a computer having a processor, memory, display, and input/output devices, the method comprising the steps of:
(a) creating records of kallikrein 5 and optionally other breast or ovarian cancer markers isolated from a sample suspected of containing breast or ovarian cancer cells or tissue;
(b) providing a database comprising records of data comprising kallikrein 5 and optionally other breast or ovarian cancer markers; and
(c) using a code mechanism for applying queries based upon a desired selection criteria to the data file in the database to produce reports of records of step (a) which provide a match of the desired selection criteria of the database of step (b) the presence of a match being a positive indication that the markers of step (a) have been isolated from cells or tissue that are breast or ovarian cancer cells or tissue.

The invention contemplates a business method for determining whether a subject has breast or ovarian cancer or a pre-disposition to breast or ovarian cancer comprising: (a) receiving phenotypic information on the subject and information on kallikrein 5 and optionally other breast or ovarian cancer markers associated with samples from the subject; (b) acquiring information from a network corresponding to kallikrein 5 and optionally other markers; and (c) based on the phenotypic information, information on kallikrein 5 and optionally other markers, and acquired information, determining whether the subject has breast or ovarian cancer or a pre-disposition to breast or ovarian cancer; and (d) optionally recommending treatment for the breast or ovarian cancer or pre-condition.

In an aspect of the invention, the computer systems, components, and methods described herein are used to monitor disease or determine the stage of disease.

Imaging Methods

Antibodies specific for kallikrein 5 may also be used in imaging methodologies in the management of breast and ovarian cancer. The invention provides a method for imaging tumors associated with one or more kallikreins, preferably kallikreins associated with breast or ovarian cancer, most preferably kallikrein 5 and optionally one or more kallikreins specifically selected from hK4, hK6, hK7, hK8, hK9, hK10, hK11, hK13, hK14, and hK15, in particular hK4, hK6, hK8, hK10, and hK11.

In an embodiment, a method of the invention comprises administering to a tissue of a subject with breast or ovarian cancer imaging agents that carry imagining labels and are capable of targeting or binding to kallikrein 5 and optionally other breast or ovarian cancer markers. The agent is allowed to incubate and bind to the kallikrein 5 and optionally other markers.

In another embodiment the method is an in vivo method and a subject or patient is administered one or more imaging agents that carry an imaging label and are capable of targeting or binding to kallikrein 5 and optionally other breast or ovarian cancer markers. The imaging agent is allowed to incubate in vivo and bind to kallikrein 5 and optionally other markers associated with a tumor, preferably breast or ovarian tumors. The presence of the label is localized to the breast or ovarian cancer, and the localized label is detected using imaging devices known to those skilled in the art.

The invention also contemplates imaging methods described herein using multiple markers for breast or ovarian cancer. For example, a method for imaging breast or ovarian cancer may further comprise injecting the patient with one or more of an agent that binds to human stratum corneum chymotryptic enzyme (HSCCE), haptoglobin alpha, osteopontin, kallikrein 2, kallikrein 3, kallikrein 4, kallikrein 6, kallikrein 7, kallikrein 8, kallikrein 9, kallikrein 10, kallikrein 11, kallikrein 13, kallikrein 14, kallikrein 15, CA125, CA15-3, CA19-9, OVX1, lysophosphatidic acid (LPA) or carcinoembryonic antigen (CEA). Preferably each agent is labeled so that it can be distinguished during the imaging.

The imaging agent may be an antibody or chemical entity that recognizes kallikrein 5 and optionally other breast or ovarian cancer markers. In an aspect of the invention the imaging agent is a polyclonal antibody or monoclonal antibody, or fragments thereof, or constructs thereof including but not limited to, single chain antibodies, bifunctional antibodies, molecular recognition units, and peptides or entities that mimic peptides. The antibodies specific for kallikrein 5 and optionally other breast or ovarian cancer markers used in the methods of the invention may be obtained from scientific or commercial sources, or isolated native proteins or recombinant proteins may be utilized to prepare antibodies etc. as described herein.

An imaging agent may be a peptide that mimics the epitope for an antibody specific for a marker (e.g. kallikrein 5) and binds to the marker. The peptide may be produced on a commercial synthesizer using conventional solid phase chemistry. By way of example, a peptide may be prepared that includes either tyrosine lysine, or phenylalanine to which N₂S₂ chelate is complexed (See U.S. Pat. No. 4,897,255). The anti-kallikrein peptide conjugate is then combined with a radiolabel (e.g. sodium ^{99m}Tc pertechnetate or sodium ¹⁸⁸Re perrhenate) and it may be used to locate a kallikrein producing tumor.

An imaging agent carries a label to image kallikrein 5 and optionally other markers. The agent may be labelled for use in radionuclide imaging. In particular, the agent may be directly or indirectly labelled with a radioisotope. Examples of radioisotopes that may be used in the present invention are the following: ²⁷⁷Ac, ²¹¹At, ¹²⁸Ba, ¹³¹Ba, ⁷Be, ²⁰⁴Bi, ²⁰⁵Bi, ²⁰⁶Bi, ⁷⁶Br, ⁷⁷Br, ⁸²Br, ¹⁰⁹Cd, ⁴⁷Ca, ¹¹C, ¹⁴C, ³⁶Cl, ⁴⁸Cr, ⁵¹Cr, ⁶²Cu, ⁶⁴Cu, ⁶⁷Cu, ¹⁶⁵Dy, ¹⁵⁵Eu, ¹⁸F, ¹⁵³Gd, ⁶⁶Ga, ⁶⁷Ga, ⁶⁸Ga, 72Ga, ¹⁹⁸Au, 3H, ¹⁶⁶Ho, ¹¹¹In, ^{113m}In, ^{115m}In, ¹²³I, ¹²⁵I, ¹³¹I, ¹⁸⁹Ir, ^{191m}Ir, ¹⁹²Ir, ¹⁹⁴Ir, ⁵²Fe, ⁵⁵Fe, ⁵⁹Fe, ¹⁷⁷Lu, ¹⁵O, ¹⁹¹m-¹⁹¹Os, ¹⁰⁹Pd, ³²P, ³³P, ⁴²K, ²²⁶Ra, ¹⁸⁶Re, ¹⁸⁸Re, ^{82m}Rb, ¹⁵³Sm, ⁴⁶Sc, ⁴⁷Sc, ⁷²Se, ⁷⁵Se, ¹⁰⁵Ag, ²²Na, ²⁴Na, ⁸⁹Sr, ³⁵S, ³⁸S, ¹⁷⁷Ta, ⁹⁶Tc, ^{99m}Tc, ²⁰¹Tl, ²⁰²Tl, ¹¹³Sn, ^{117m}Sn, ¹²¹Sn, ¹⁶⁶Yb, ¹⁶⁹Yb, ¹⁷⁵Yb, ⁸⁸Y, ⁹⁰Y, ⁶²Zn and ⁶⁵Zn. Preferably the radioisotope is ¹³¹I, ¹²⁵I, ¹²³I, ¹¹¹I, ^{99m}Tc, ⁹⁰Y, ¹⁸⁶Re, ¹⁸⁸Re, ³²P, ¹⁵³Sm, ⁶⁷Ga, ²⁰¹Tl, ⁷⁷Br, or ¹⁸F, and is imaged with a photoscanning device.

Procedures for labeling biological agents with the radioactive isotopes are generally known in the art. U.S. Pat. No. 4,302,438 describes tritium labeling procedures. Procedures for iodinating, tritium labeling, and ³⁵S labeling especially adapted for-murine monoclonal antibodies are described by Goding, J. W. (supra, pp 124-126) and the references cited therein. Other procedures for iodinating biological agents, such as antibodies, binding portions thereof, probes, or ligands, are described in the scientific literature (see Hunter and Greenwood, Nature 144:945 (1962), David et al., Biochemistry 13:1014-1021 (1974), and U.S. Pat. Nos. 3,867,517 and 4,376,110). Iodinating procedures for agents are described by Greenwood, F. et al., Biochem. J. 89:114-123 (1963); Marchalonis, J., Biochem. J. 113:299-305 (1969); and Morrison, M. et al., Immunochemistry, 289-297 (1971). ^{99m}Tc-labeling procedures are described by Rhodes, B. et al. in Burchiel, S. et al. (eds.), Tumor Imaging: The Radioimmunochemical Detection of Cancer, New York: Masson 111-123 (1982) and the references cited therein. Labelling of antibodies or fragments with technetium-99m are also described for example in U.S. Pat. No. 5,317,091, U.S. Pat. No. 4,478,815, U.S. Pat. No. 4,478,818, U.S. Pat. No. 4,472,371, U.S. Pat. No. Re 32,417, and U.S. Pat. No. 4,311,688. Procedures suitable for ¹¹¹In-labeling biological agents are described by Hnatowich, D. J. et al., J. Immul. Methods, 65:147-157 (1983), Hnatowich, D. et al., J. Applied Radiation, 35:554-557 (1984), and Buckley, R. G. et al., F.E.B.S. 166:202-204 (1984).

An agent may also be labeled with a paramagnetic isotope for purposes of an in vivo method of the invention. Examples of elements that are useful in magnetic resonance imaging include gadolinium, terbium, tin, iron, or isotopes thereof (See, for example, Schaefer et al., (1989) JACC 14, 472-480; Shreve et al., (1986) Magn. Reson. Med. 3, 336-340; Wolf, G. L., (1984) Physiol. Chem. Phys. Med. NMR 16, 93-95; Wesbey et al., (1984) Physiol. Chem. Phys. Med. NMR 16, 145-155; Runge et al., (1984) Invest. Radiol. 19, 408-415 for discussions on in vivo nuclear magnetic resonance imaging.)

In the case of a radiolabeled agent, the agent may be administered to the patient, it is localized to the tumor having a kallikrein with which the agent binds, and is detected or “imaged” in vivo using known techniques such as radionuclear scanning using e.g., a gamma camera or emission tomography. [See for example A. R. Bradwell et al., “Developments in Antibody Imaging”, Monoclonal Antibodies for Cancer Detection and Therapy, R. W. Baldwin et al., (eds.), pp. 65-85 (Academic Press 1985)]. A positron emission transaxial tomography scanner, such as designated Pet VI located at Brookhaven National Laboratory, can also be used where the radiolabel emits positrons (e.g., ¹¹C, ¹⁸F, ¹⁵O, and ¹³N).

Whole body imaging techniques using radioisotope labeled agents can be used for locating both primary tumors and tumors which have metastasized. Antibodies specific for kallikreins, or fragments thereof having the same epitope specificity, are bound to a suitable radioisotope, or a combination thereof, and administered parenterally. The bio-distribution of the label can be monitored by scintigraphy, and accumulations of the label are related to the presence of breast or ovarian cancer cells. Whole body imaging techniques are described in U.S. Pat. Nos. 4,036,945 and 4,311,688. Other examples of agents useful for diagnosis and therapeutic use that can be coupled to antibodies and antibody fragments include metallothionein and fragments (see, U.S. Pat. No. 4,732,864). These agents are useful in diagnosis staging and visualization of cancer, in particular breast or ovarian cancer, so that surgical and/or radiation treatment protocols can be used more efficiently.

An imaging agent may carry a bioluminescent or chemiluminescent label. Such labels include polypeptides known to be fluorescent, bioluminescent or chemiluminescent, or, that act as enzymes on a specific substrate (reagent), or can generate a fluorescent, bioluminescent or chemiluminescent molecule. Examples of bioluminescent or chemiluminescent labels include luciferases, aequorin, obelin, mnemiopsin, berovin, a phenanthridinium ester, and variations thereof and combinations thereof. A substrate for the bioluminescent or chemiluminescent polypeptide may also be utilized in a method of the invention. For example, the chemiluminescent polypeptide can be luciferase and the reagent luciferin. A substrate for a bioluminescent or chemiluminescent label can be administered before, at the same time (e.g., in the same formulation), or after administration of the agent.

An imaging agent may comprise a paramagnetic compound, such as a polypeptide chelated to a metal, e.g., a metalloporphyrin. The paramagnetic compound may also comprise a monocrystalline nanoparticle, e.g., a nanoparticle comprising a lanthanide (e.g., Gd) or iron oxide; or, a metal ion comprising a lanthanide. “Lanthanides” refers to elements of atomic numbers 58 to 70, a transition metal of atomic numbers 21 to 29, 42 or 44, a Gd(III), a Mn(II), or an element comprising an Fe element. Paramagnetic compounds can also comprise a neodymium iron oxide (NdFeO.sub.3) or a dysprosium iron oxide (DyFeO.sub.3). Examples of elements that are useful in magnetic resonance imaging include gadolinium, terbium, tin, iron, or isotopes thereof. (See, for example, Schaefer et al.; (1989) JACC 14, 472-480; Shreve et al., (1986) Magn. Reson. Med. 3, 336-340; Wolf, G L., (1984) Physiol. Chem. Phys. Med. NMR 16, 93-95; Wesbey et al., (1984) Physiol. Chem. Phys. Med. NMR 16, 145-155; Runge et al., (1984) Invest. Radiol. 19, 408-415 for discussions on in vivo nuclear magnetic resonance imaging.)

An image can be generated in a method of the invention by computer assisted tomography (CAT), magnetic resonance spectroscopy (MRS) image, magnetic resonance imaging (MRI), positron emission tomography (PET), single-photon emission computed tomography (SPECT), or bioluminescence imaging (BLI) or equivalent.

Computer assisted tomography (CAT) and computerized axial tomography (CAT) systems and devices well known in the art can be utilized in the practice of the present invention. ( See, for example, U.S. Pat. Nos. 6,151,377; 5,946,371; 5,446,799; 5,406,479; 5,208,581; 5,109,397). The invention may also utilize animal imaging modalities, such as MicroCAT™ (ImTek, Inc.).

Magnetic resonance imaging (MRI) systems and devices well known in the art can be utilized in the practice of the present invention. In magnetic resonance methods and devices, a static magnetic field is applied to a tissue or a body in order to define an equilibrium axis of magnetic alignment in a region of interest. A radio-frequency field is then applied to the region in a direction orthogonal to the static magnetic field direction to excite magnetic resonance in the region. The resulting radio frequency signals are then detected and processed, and the exciting radio frequency field is applied. The resulting signals are detected by radio-frequency coils that are placed adjacent to the tissue or area of the body of interest. (For a description of MRI methods and devices see, for example, U.S. Pat. Nos. 6,151,377; 6,144,202; 6,128,522; 6,127,825; 6,121,775; 6,119,032; 6,115,446; 6,111,410; 602,891; 5,555,251; 5,455,512; 5,450,010; 5,378,987; 5,214,382; 5,031,624; 5,207,222; 4,985,678; 4,906,931; 4,558,279). MRI and supporting devices are commercially available for example, from Bruker Medical GMBH; Caprius; Esaote Biomedica; Fonar; GE Medical Systems (GEMS); Hitachi Medical Systems America; Intermagnetics General Corporation; Lunar Corp.; MagneVu; Marconi Medicals; Philips Medical Systems; Shimadzu; Siemens; Toshiba America Medical Systems; including imaging systems, by, e.g., Silicon Graphics. The invention may also utilize animal imaging modalities such as micro-MRIs.

Positron emission tomography imaging (PET) systems and devices well known in the art can be utilized in the practice of the present invention. For example, a method of the invention may use the system designated Pet VI located at Brookhaven National Laboratory. For descriptions of PET systems and devices see, for example, U.S. Pat. Nos. 6,151,377; 6,072,177; 5,900,636; 5,608,221; 5,532,489; 5,272,343; 5,103,098. Animal imaging modalities such as micro-PETs (Corcorde Microsystems, Inc.) can also be used in the invention.

Single-photon emission computed tomography (SPECT) systems and devices well known in the art can be utilized in the practice of the present invention. (See, for example, U.S. Pat. Nos. 6,115,446; 6,072,177; 5,608,221; 5,600,145; 5,210,421; 5,103,098.) The methods of the invention may also utilize animal imaging modalities, such as micro-SPECTs.

Bioluminescence imaging includes bioluminescence, fluorescence or chemiluminescence or other photon detection systems and devices that are capable of detecting bioluminescence, fluorescence or chemiluminescence. Sensitive photon detection systems can be used to detect bioluminescent and fluorescent proteins externally; see, for example, Contag (2000) Neoplasia 2:41-52; Zhang (1994) Clin. Exp. Metastasis 12:87-92. The methods of the invention can be practiced using any such photon detection device, or variation or equivalent thereof, or in conjunction with any known photon detection methodology, including visual imaging. By way of example, an intensified charge-coupled device (ICCD) camera coupled to an image processor may be used in the present invention. (See, e.g., U.S. Pat. No. 5,650,135). Photon detection devices are also commercially available from Xenogen, Hamanatsue.

Screening Methods

The invention also contemplates methods for evaluating test agents or compounds for their ability to inhibit breast or ovarian cancer or potentially contribute to breast or ovarian cancer. Test agents and compounds include but are not limited to peptides such as soluble peptides including Ig-tailed fusion peptides, members of random peptide libraries and combinatorial chemistry-derived molecular libraries made of D- and/or L-configuration amino acids, phosphopeptides (including members of random or partially degenerate, directed phosphopeptide libraries), antibodies [e.g. polyclonal, monoclonal, humanized, anti-idiotypic, chimeric, single chain antibodies, fragments, (e.g. Fab, F(ab)₂, and Fab expression library fragments, and epitope-binding fragments thereof)], and small organic or inorganic molecules. The agents or compounds may be endogenous physiological compounds or natural or synthetic compounds.

The invention provides a method for assessing the potential efficacy of a test agent for inhibiting breast or ovarian cancer in a patient, the method comprising comparing:
(a) levels of kallikrein 5 and optionally other breast or ovarian cancer markers in a first sample obtained from a patient and exposed to the test agent; and
(b) levels of kallikrein 5 and optionally other markers in a second sample obtained from the patient, wherein the sample is not exposed to the test agent, wherein a significant difference in the levels of expression of kallikrein 5 and optionally the other markers in the first sample, relative to the second sample, is an indication that the test agent is potentially efficacious for inhibiting breast or ovarian cancer in the patient.

The first and second samples may be portions of a single sample obtained from a patient or portions of pooled samples obtained from a patient.

In an embodiment, the levels of expression of kallikrein 5 in the first sample are significantly lower relative to the second sample.

In an aspect, the invention provides a method of selecting an agent for inhibiting breast or ovarian cancer in a patient comprising:
(a) obtaining a sample from the patient;
(b) separately maintaining aliquots of the sample in the presence of a plurality of test agents;
(c) comparing kallikrein 5 and optionally other breast or ovarian cancer markers, in each of the aliquots; and
(d) selecting one of the test agents which alters the levels of kallikrein 5 and optionally other breast or ovarian cancer markers in the aliquot containing that test agent, relative to other test agents.

In an embodiment, the levels of kallikrein 5 are significantly lower in the presence of the selected test agent.

Still another aspect of the present invention provides a method of conducting a drug discovery business comprising:
(a) providing one or more methods or assay systems for identifying agents that inhibit breast or ovarian cancer in a patient;
(b) conducting therapeutic profiling of agents identified in step (a), or further analogs thereof, for efficacy and toxicity in animals; and
(c) formulating a pharmaceutical preparation including one or more agents identified in step (b) as having an acceptable therapeutic profile.

In certain embodiments, the subject method can also include a step of establishing a distribution system for distributing the pharmaceutical preparation for sale, and may optionally include establishing a sales group for marketing the pharmaceutical preparation.

The invention also contemplates a method of assessing the potential of a test compound to contribute to breast or ovarian cancer comprising:
(a) maintaining separate aliquots of cells or tissues from a patient with breast or ovarian cancer in the presence and absence of the test compound; and
(b) comparing kallikrein 5 and optionally other breast or ovarian cancer markers in each of the aliquots.

A significant difference between the levels of the markers in the aliquot maintained in the presence of (or exposed to) the test compound relative to the aliquot maintained in the absence of the test compound, indicates that the test compound possesses the potential to contribute to breast or ovarian cancer. In an embodiment, the levels of kallikrein 5 are higher in the presence of the test compound.

Kits

The invention contemplates kits for carrying out the methods of the invention. Such kits typically comprise two or more components required for performing a diagnostic assay. Components include but are not limited to compounds, reagents, containers, and/or equipment.

The methods described herein may be performed by utilizing pre-packaged diagnostic kits comprising at least binding agents (e.g. antibodies) described herein, which may be conveniently used, e.g., in clinical settings, to screen and diagnose patients, and to screen and identify those individuals afflicted with or exhibiting a predisposition to breast or ovarian cancer.

In an embodiment, a container with a kit comprises binding agents as described herein. By way of example, the kit may contain antibodies specific for kallikrein 5 and optionally other breast or ovarian cancer markers, antibodies against the antibodies labelled with enzymes, and substrates for the enzymes. The kit may also contain microtiter plate wells, standards, assay diluent, wash buffer, adhesive plate covers, and/or instructions for carrying out a method of the invention using the kit.

In an aspect of the invention, the kit includes antibodies or antibody fragments which bind specifically to an epitope of kallikrein 5 and optionally other breast or ovarian cancer markers, and means for detecting binding of the antibodies to an epitope associated with breast or ovarian cancer, either as concentrates (including lyophilized compositions), which may be further diluted prior to use or at the concentration of use, where the vials may include one or more dosages. Where the kits are intended for in vivo use, single dosages may be provided in sterilized containers, having the desired amount and concentration of agents. Containers that provide a formulation for direct use, usually do not require other reagents, as for example, where the kit contains radiolabelled antibody preparations for in vivo imaging.

The reagents suitable for applying the screening methods of the invention to evaluate compounds may be packaged into convenient kits described herein providing the necessary materials packaged into suitable containers.

Thus, the invention relates to a kit for assessing the suitability of each of a plurality of test compounds for inhibiting endocrine cancer in a patient. The kit comprises reagents for assessing kallikrein 5 and optionally a plurality of test agents or compounds.

The invention contemplates a kit for assessing the presence of endocrine cancer cells, wherein the kit comprises antibodies specific for kallikrein 5, and optionally antibodies specific for other markers associated with endocrine cancer.

Additionally the invention provides a kit for assessing the potential of a test compound to contribute to endocrine cancer. The kit comprises endocrine cancer cells and reagents for assessing kallikrein 5, and optionally other markers associated with endocrine cancer.

Therapeutic Applications

Kallikrein 5 is expressed or overexpressed in breast and ovarian cancer and it thus can be a target for cancer immunotherapy. Such immunotherapeutic methods include the use of antibody therapy, in vivo vaccines, and ex vivo immunotherapy approaches.

In one aspect, the invention provides kallikrein 5 antibodies that may be used systemically to treat cancer, such as ovarian and breast cancer. Preferably antibodies are used that target the tumor cells but not the surrounding non-tumor cells and tissue. Thus, the invention provides a method of treating a patient susceptible to, or having a cancer that expresses kallikrein 5, comprising administering to the patient an effective amount of an antibody that binds specifically to kallikrein 5. In another aspect, the invention provides a method of inhibiting the growth of tumor cells expressing kallikrein 5, comprising administering to a patient an antibody which binds specifically to kallikrein 5 in an amount effective to inhibit growth of the tumor cells. Kallikrein 5 antibodies may also be used in a method for selectively inhibiting the growth of or killing a cell expressing kallikrein 5 comprising reacting a kallikrein 5 antibody immunoconjugate or immunotoxin with the cell in an amount sufficient to inhibit the growth of or kill the cell.

By way of example, unconjugated kallikrein 5 antibody may be introduced into a patient such that the antibody binds to kallikrein 5 expressing cancer cells and mediates growth inhibition of such cells (including the destruction thereof), and the tumor, by mechanisms which may include complement-mediated cytolysis, antibody-dependent cellular cytotoxicity, altering the physiologic function of kallikrein 5, and/or the inhibition of ligand binding or signal transduction pathways. In addition to unconjugated kallikrein 5 antibodies, kallikrein 5. antibodies conjugated to therapeutic agents (e.g. immunoconjugates) may also be used therapeutically to deliver the agent directly to kallikrein 5 expressing tumor cells and thereby destroy the tumor. Examples of such agents include abrin, ricin A, Pseudomonas exotoxin, or diphtheria toxin; proteins such as tumor necrosis factor, alpha-interferon, beta-interferon, nerve growth factor; platelet derived growth factor, tissue plasminogen activator; and biological response modifiers such as lymphokines, interleukin-1, intarleukin-2, interleukin-6, granulocyte macrophage colony stimulating factor, granulocyte colony stimulating factor, or other growth factors.

Cancer immunotherapy using kallikrein 5 antibodies may utilize the various approaches that have been successfully employed for cancers, including but not limited to colon cancer (Arlen et al., 1998, Crit Rev Immunol 18: 133-138), multiple myeloma (Ozaki et al., 1997, Blood 90: 3179-3186; Tsunenati et al., 1997, Blood 90: 2437-2444), gastric cancer (Kasprzyk et al., 1992, Cancer Res 52: 2771-2776), B-cell lymphoma (Funakoshi et al., 1996, J Immunther Emphasis Tumor Immunol 19: 93-101), leukemia (Zhong et al., 1996, Leuk Res 20: 581-589), colorectal cancer (Moun et al., 1994, Cancer Res 54: 6160-6166); Velders et al., 1995, Cancer Res 55: 4398-4403), and breast cancer (Shepard et al., 1991, J Clin Immunol 11: 117-127).

In the practice of the method of the invention, anti-kallikrein 5 antibodies capable of inhibiting the growth of cancer cells expressing kallikrein 5 are administered in a therapeutically effective amount to cancer patients whose tumors express or overexpress kallikrein 5. The invention may provide a specific, effective and long-needed treatment for ovarian or breast cancer. The antibody therapy methods of the invention may be combined with other therapies including but not limited to chemotherapy and radiation.

Patients may be evaluated for the presence and level of kallikrein 5 expression and overexpression in tumors, preferably using immunohistochemical assessments of tumor tissue, quantitative kallikrein 5 imaging as described herein, or other techniques capable of reliably indicating the presence and degree of kallikrein 5 expression. Immunohistochemical analysis of tumor biopsies or surgical specimens may be employed for this purpose.

Anti-kallikrein 5 antibodies useful in treating cancer include those that are capable of initiating a potent immune response against the tumor and those that are capable of direct cytotoxicity. In this regard, anti-kallikrein 5 antibodies may elicit tumor cell lysis by either complement-mediated or antibody-dependent cell cytotoxicity (ADCC) mechanisms, both of which require an intact Fc portion of the immunoglobulin molecule for interaction with effector cell Fc receptor sites or complement proteins. In addition, anti-kallikrein 5 antibodies that exert a direct biological effect on tumor growth are useful in the practice of the invention. Such antibodies may not require the complete immunoglobulin to exert the effect. Potential mechanisms by which such directly cytotoxic antibodies may act include inhibition of cell growth, modulation of cellular differentiation, modulation of tumor angiogenesis factor profiles, and the induction of apoptosis. The mechanism by which a particular anti-kallikrein 5 antibody exerts an anti-tumor effect may be evaluated using any number of in vitro assays designed to determine ADCC, antibody-dependent macrophage-mediated cytotoxicity (ADMMC), complement-mediated cell lysis, and others known in the art.

The anti-tumor activity of a particular anti-kallikrein 5 antibody, or combination of anti-kallikrein 5 antibodies, may be evaluated in vivo using a suitable animal model. Xenogenic cancer models, wherein human cancer explants or passaged xenograft tissues are introduced into immune compromised animals, such as nude or SCID mice, may be employed.

The methods of the invention contemplate the administration of single anti-kallikrein 5 antibodies as well as combinations, or “cocktails”, of different individual antibodies such as those recognizing different epitopes or other kallikreins or breast or ovarian cancer markers. Such cocktails may have certain advantages inasmuch as they contain antibodies that bind to different epitopes or kallikreins and/or exploit different effector mechanisms or combine directly cytotoxic antibodies with antibodies that rely on immune effector functionality. Such antibodies in combination may exhibit synergistic therapeutic effects. In addition, the administration of anti-kallikrein 5 antibodies may be combined with other therapeutic agents, including but not limited to chemotherapeutic agents, androgen-blockers, and immune modulators (e.g., IL2, GM-CSF). The anti-kallikrein 5 antibodies may be administered in their “naked” or unconjugated form, or may have therapeutic agents conjugated to them.

The anti-kallikrein 5 antibodies used in the practice of the method of the invention may be formulated into pharmaceutical compositions comprising a carrier suitable for the desired delivery method. Suitable carriers include any material which when combined with the antibodies retains the anti-tumor function of the antibody and is non-reactive with the subject's immune systems. Examples include any of a number of standard pharmaceutical carriers such as sterile phosphate buffered saline solutions, bacteriostatic water, and the like (see, generally, Remington's Pharmaceutical Sciences 16.sup.th Edition, A. Osal., Ed., 1980).

Anti-kallikrein 5 antibody formulations may be administered via any route capable of delivering the antibodies to the tumor site. Routes of administration include, but are not limited to, intravenous, intraperitoneal, intramuscular, intratumor, intradermal, and the like. Preferably, the route of administration is by intravenous injection. Anti-kallikrein 5 antibody preparations may be lyophilized and stored as a sterile powder, preferably under vacuum, and then reconstituted in bacteriostatic water containing, for example, benzyl alcohol preservative, or in sterile water prior to injection.

Treatment will generally involve the repeated administration of the antibody preparation via an acceptable route of administration such as intravenous injection (IV), at an effective dose. Dosages will depend upon various factors generally appreciated by those of skill in the art, including the type of cancer and the severity, grade, or stage of the cancer, the binding affinity and half life of the antibodies used, the degree of kallikrein 5 expression in the patient, the extent of circulating kallikrein 5 antigen, the desired steady-state antibody concentration level, frequency of treatment, and the influence of any chemotherapeutic agents used in combination with the treatment method of the invention.

Daily doses may range from about 0.1 to 100 mg/kg. Doses in the range of 10-500 mg antibodies per week may be effective and well tolerated, although even higher weekly doses may be appropriate and/or well tolerated. A determining factor in defining the appropriate dose is the amount of a particular antibody necessary to be therapeutically effective in a particular context. Repeated administrations may be required to achieve tumor inhibition or regression. Direct administration of kallikrein 5 antibodies is also possible and may have advantages in certain situations.

Patients may be evaluated for serum kallikrein 5 in order to assist in the determination of the most effective dosing regimen and related factors. The kallikrein 5 assay methods described herein, or similar assays, may be used for quantitating circulating kallikrein 5 levels in patients prior to treatment. Such assays may also be used for monitoring throughout therapy, and may be useful to gauge therapeutic success in combination with evaluating other parameters such as serum kallikrein 5 levels.

The invention further provides vaccines formulated to contain a kallikrein 5 protein or fragment thereof. The use in anti-cancer therapy of a tumor antigen in a vaccine for generating humoral and cell-mediated immunity is well known and, for example, has been employed in prostate cancer using human PSMA and rodent PAP immunogens (Hodge et al., 1995, Int. J. Cancer 63: 231-237; Fong et al., 1997, J. Immunol. 159: 3113-3117). These methods can be practiced by employing a kallikrein 5 protein, or fragment thereof, or a kallikrein 5-encoding nucleic acid molecule and recombinant vectors capable of expressing and appropriately presenting the kallikrein 5 immunogen.

By way of example, viral gene delivery systems may be used to deliver a kallikrein 5 encoding nucleic acid molecule (i.e. KLK5). Various viral gene delivery systems which can-be used in the practice of this aspect of the invention include, but are not limited to, vaccinia, fowlpox, canarypox, adenovirus, influenza, poliovirus, adeno-associated virus, lentivirus, and sindbus virus (Restifo, 1996, Curr. Opin. Immunol. 8: 658-663). Non-viral delivery systems may also be employed by using naked DNA encoding a kallikrein 5 protein or fragment thereof introduced into the patient (e.g., intramuscularly) to induce an anti-tumor response.

Various ex vivo strategies may also be employed. One approach involves the use of cells to present kallikrein 5 antigen to a patient's immune system. For example, autologous dendritic cells which express MHC class I and II, may be pulsed with kallikrein 5 or peptides thereof that are capable of binding to MHC molecules, to thereby stimulate cancer (e.g. breast or ovarian cancer) patients' immune systems (See, for example, Tjoa et al., 1996, Prostate 28: 65-69; Murphy et al., 1996, Prostate 29: 371-380).

Anti-idiotypic anti-kallikrein 5 antibodies can also be used in anti-cancer therapy as a vaccine for inducing an immune response to cells expressing a kallikrein 5 protein. The generation of anti-idiotypic antibodies is well known in the art and can readily be adapted to generate anti-idiotypic anti-kallikrein 5 antibodies that mimic an epitope on a kallikrein 5 protein (see, for example, Wagner et al., 1997, Hybridoma 16: 33-40; Foon et al., 1995, J Clin Invest 96: 334-342; Herlyn et al., 1996, Cancer Immunol Immunother 43: 65-76). Such an antibody can be used in anti-idiotypic therapy as presently practiced with other anti-idiotypic antibodies directed against tumor antigens.

Genetic immunization methods may be utilized to generate prophylactic or therapeutic humoral and cellular immune responses directed against cancer cells expressing kallikrein 5. Using the kallikrein 5 encoding DNA molecules, constructs comprising DNA encoding a kallikrein 5 protein/immunogen and appropriate regulatory sequences may be injected directly into muscle or skin of an individual, such that the cells of the muscle or skin take-up the construct and express the encoded kallikrein 5 protein/immunogen. The kallikrein 5 protein/immunogen may be expressed as a cell surface protein or be secreted. Expression of the kallikrein 5 protein/immunogen results in the generation of prophylactic or therapeutic humoral and cellular immunity against the cancer. Various prophylactic and therapeutic genetic immunization techniques known in the art may be used.

The invention further provides methods for inhibiting cellular activity (e.g., cell proliferation, activation, or propagation) of a cell expressing kallikrein 5. This method comprises reacting immunoconjugates of the invention (e.g., a heterogeneous or homogenous mixture) with the cell so that the kallikrein 5 proteins form a complex with the immunoconjugates. A subject with a neoplastic or preneoplastic condition can be treated when the inhibition of cellular activity results in cell death.

In another aspect, the invention provides methods for selectively inhibiting a cell expressing kallikrein 5 by reacting any one or a combination of the immunoconjugates of the invention with the cell in an amount sufficient to inhibit the cell. Amounts include those that are sufficient to kill the cell or sufficient to inhibit cell growth or proliferation.

Kallikrein 5 and fragments thereof, and agents identified using a method of the invention may be used in the treatment of breast or ovarian cancer in a subject. These polypeptides and agents may be formulated into compositions for administration to subjects suffering from breast or ovarian cancer. Therefore, the present invention also relates to a composition comprising kallikrein 5 or a fragment thereof, or an agent identified using a method of the invention, and a pharmaceutically acceptable carrier, excipient or diluent. A method for treating or preventing breast or ovarian cancer in a subject is also provided comprising administering to a patient in need thereof kallikrein 5 or an agent identified in accordance with a method of the invention, or a composition of the invention.

The invention further provides a method of inhibiting breast or ovarian cancer in a patient comprising:
(a) obtaining a sample comprising diseased cells from the patient;
(b) separately maintaining aliquots of the sample in the presence of a plurality of test agents;
(c) comparing levels of kallikrein 5 in each aliquot;
(d) administering to the patient at least one of the test agents which alters the levels of the kallikrein 5 in the aliquot containing that test agent, relative to the other test agents.

In an embodiment, a test agent that decreases the levels of kallikrein 5 in an aliquot is administered to the patient.

The active substance may be administered in a convenient manner such as by injection (subcutaneous, intravenous, etc.), oral administration, inhalation, transdermal application, or rectal administration. Depending on the route of administration, the active substance may be coated in a material to protect the substance from the action of enzymes, acids and other natural conditions that may inactivate the substance. Solutions of an active compound as a free base or pharmaceutically acceptable salt can be prepared in an appropriate solvent with a suitable surfactant. Dispersions may be prepared in glycerol, liquid polyethylene glycols, and mixtures thereof, or in oils.

The compositions described herein can be prepared by per se known methods for the preparation of pharmaceutically acceptable compositions which can be administered to subjects, such that an effective quantity of the active substance is combined in a mixture with a pharmaceutically acceptable vehicle. Suitable vehicles are described, for example, in Remington's Pharmaceutical Sciences (Remington's Pharmaceutical Sciences, Mack Publishing Company, Easton, Pa., USA 1985). On this basis, the compositions include, albeit not exclusively, solutions of the active substances in association with one or more pharmaceutically acceptable vehicles or diluents, and contained in buffered solutions with a suitable pH and iso-osmotic with the physiological fluids.

The compositions are indicated as therapeutic agents either alone or in conjunction with other therapeutic agents or other forms of treatment. The compositions of the invention may be administered concurrently, separately, or sequentially with other therapeutic agents or therapies.

The therapeutic activity of compositions and agents/compounds identified using a method of the invention and may be evaluated in vivo using a suitable animal model.

The following non-limiting example is illustrative of the present invention:

### EXAMPLE 1

Materials and Methods

Production and Purification of Recombinant hK5

Cloning of KLK5 cDNA into a Yeast Expression System.

Recombinant hK5 was produced using the Pichia pastoris yeast expression system (Invitrogen, Carlsbad, Calif., USA). Two primers were designed to amplify the KLK5 cDNA sequence: the forward primer was 5′ AGC AGC CGA TCG ATC AAT GGA TCC GAC TG 3′ [SEQ ID NO. 3] and the reverse primer was 5′ CTG AGT CCT GGG ATG TCT AGA GAG TTG GC 3′ [SEQ ID NO. 4]. Human breast cDNA was used as a template. PCR was carried out in a 50 μL reaction mixture, containing 1 μL of cDNA, 10 mM Tris-HCl (pH 8.3), 50 mM KCl, 1.5 mM MgCl₂, 10 μM deoxynucleoside triphosphates, 30 μM primers, and 0.75 μL of Expand Long Template PCR polymerase mix (Roche Diagnostics, Mannheim, Germany), using an Eppendorf master cycler. The PCR conditions were 94° C. for 2 min, followed by 94° C. for 10 s, 54° C. for 30 s, 68° C. for 1 min for 40 cycles, and a final extension at 68° C. for 7 min. The PCR product was then cloned into the yeast expression vector pPICZα using standard procedures (11). The sequence of the construct was confirmed with an automated DNA sequencer.

Production of hK5 in Yeast.

The pPICZαC vector containing the mature KLK5 cDNA sequence was introduced into the yeast strains X-33, KM71 and GS115. A stable clone was selected from the KM71 strain following the manufacturer's recommendations (Invitrogen). hK5 was produced by growing the stable yeast clone in a medium containing 10 g/L yeast extract, 20 g/L peptone, 100 mmol/L potassium phosphate (pH 6.0), 13.4 g/L yeast nitrogen base and 40 mg/L biotin in a 30° C. shaking incubator (250 rpm). hK5 production was induced with 1% methanol/day over 6 days. The cells were subsequently spun down, and the supernatant collected. An hK5 rabbit polyclonal peptide antibody (produced in-house) was used to monitor hK5 production by Western blot analysis.

Characterization of hK5 by Mass Spectrometry.

Purified hK5 was applied to polyacrylamide gels (˜1 μg) and stained with Coomassie G-250 for visualization. The stained band was subsequently excised and destained with 300 mL/L acetonitrile in 100 mM ammonium bicarbonate. The band was then reduced (10 mmol/L dithiothreitol in 50 mM ammonium bicarbonate, pH 8.3) and alkylated (50 mM iodoacetamide in 50 mmol/L ammonium bicarbonate, pH 8.3) before overnight trypsin digestion. Peptide fragments were then extracted with 50 mL/L acetic acid, evaporated dry on a Savant concentrator, and reconstituted in 10 μL of a solution of methanol-water-acetic acid (500:495:5 by volume).

All nanoelectrospray mass spectrometry experiments were conducted on a Q-star (PE/Sciex, Concord, ON, Canada) hybrid quadrupole time-of-flight instrument, for high resolution and online tandem mass spectrometry (MS-MS) experiments (12). Conventional mass spectra were obtained by operating the quadrupole in a radiofrequency-only mode while a pusher electrode was pulsed at a frequency of approximately 7 kHz to transfer all ions to the time-of-flight analyzer. MS-MS experiments on trypsin-digested peptides identified in survey scans were conducted using a nanoelectrospray source. Precursor ions were selected by the first quadrupole while a pusher electrode was pulsed (frequency approximately 7 kHz) to transfer fragment ions formed in the radiofrequency-only quadrupole cell to the time-of-flight analyzer. Mass spectral resolution was typically 9,000-10,000. A scan duration of 1 and 2 s was set for conventional and MS-MS mass spectral acquisition, respectively. Collisional activation was performed using nitrogen collision gas with typically a 30-V offset between the DC voltage of the entrance quadrupole and the radiofrequency-only quadrupole cell. Data was acquired and processed using LC Tune and Biomultiview programs from PE/Sciex.

Purification of hK5 with Cation-Exchange and Reversed-Phase Chromatography.

Recombinant hK5 was purified from yeast culture supernatants by cation-exchange chromatography using CM-Sepharose fast flow (Pharmacia Biotech, Piscataway, N.J.) and reverse-phase liquid chromatography using a C₄ column (0.45×5 cm; Vydac). The presence of hK5 in various fractions was identified by Western blotting using an anti-hK5 peptide antibody. In summary, the CM-Sepharose beads, previously activated with 1M KCl, were equilibrated in 10 mM MES buffer (pH 6.5). The yeast culture supernatant was then diluted four-fold with 10 mM MES buffer (pH 6.5), the pH was adjusted to 6.5 and applied to the column. After washing the column, hK5 was eluted with 300 mM KCl in 10 mM MES buffer (pH 6.5). Trifluoroacetic acid as ion-pairing agent was added into this eluate (final concentration, 10 mL/L), which was then loaded on a C₄ column, equilibrated with 1 mL/L trifluoroacetic acid in water. A linear gradient (1 %/min) of acetonitrile from 15% to 50% in 1 mL/L trifluoroacetic acid was then performed. The fraction containing hK5 was evaporated on a SpeedVac (Savant). The purified material was then separated by sodium dodecyl sulfate-polyacrylamide gel electrophoresis (SDS-PAGE) and stained with Coomassie Blue to assess its purity and its molecular mass. The protein concentration of the purified hK5 was determined by the bicinchoninic acid method, which uses bovine serum albumin as calibrator (Pierce Chemical Co, Rockford, Ill.).

Production of Antibodies Against hK5.

The purified recombinant hK5 protein was used as an immunogen to immunize rabbits and mice. hK5 (100 μg) was injected subcutaneously into Balb/C female mice and New Zealand white female rabbits. The protein was diluted 1:1 in complete Freund's adjuvant for the first injection and in incomplete Freund's adjuvant for the subsequent injections. Injections were repeated six times at 3-week intervals. Blood was drawn from the animals and tested for antibody generation. To test for production of anti-hK5 polyclonal antibodies in mice and rabbits, the following immunoassay was used. Sheep anti-mouse or goat anti-rabbit IgG, respectively (Jackson ImmunoResearch, West Grove, Pa., USA) was immobilized on 96-well white ELISA plates. The mouse/rabbit serum was then applied to the plates at different dilutions ranging from 1:500 to 1:50,000. After incubation (1 h) and washing, biotinylated recombinant hK5 was added to each well (5-10 ng/well). Finally, after incubation (1 h) and washing, alkaline phosphatase-conjugated streptavidin was added, incubated for 15 min, washed and the alkaline phosphatase activity was detected with time-resolved fluorescence (for details, see below).

Immunofluorometric Assay for hK5

Standard Assay Procedure.

A sandwich-type, non-competitive immunoassay (enzyme-linked immunosorbent assay, ELISA) was developed as follows: White polystyrene microtiter plates were coated with sheep anti-mouse IgG, Fc fragment-specific antibody (Jackson ImmunoResearch) by overnight incubation of 100 μL of coating antibody solution (containing 500 ng of antibody diluted in 50 mmol/L Tris buffer, pH 7.80) in each well. The plates were then washed six times with the washing buffer (9 g/L NaCl and 0.5 g/L Tween 20 in 10 mmol/L Tris buffer, pH 7.4). Mouse anti-hK5 polyclonal antiserum was diluted 2,000-fold in a general diluent (60 g/L bovine serum albumin, 50 mmol/L Tris (pH 7.80) and 0.5 g/L sodium azide), and 100 μL were applied to each well. After 2 h incubation, the plates were washed six times with washing buffer.

hK5 calibrators or samples were then pipetted into each well (50 μL/well) along with 50 μL of the general diluent and incubated for 1 h with shaking; the plates were then washed with washing buffer six times. Subsequently, 100 μL of rabbit anti-hK5 antiserum, diluted 500-fold in buffer A (containing the components of the general diluent plus 25 mL/L normal mouse serum, 100 mL/L normal goat serum, and 10 g/L bovine IgG) were applied to each well and incubated for 1 h; plates were then washed as described earlier. Finally, 100 μL/well of alkaline phosphatase-conjugated goat anti-rabbit IgG, Fc fragment-specific (Jackson ImmunoResearch), diluted 3,000-fold in buffer A were added to each well, incubated for 45 min and plates were washed as above.

Diflunisal phosphate (100 μL of a 1 mmol/L solution) in substrate buffer (0.1 mol/L Tris, pH 9.1, 0.1 mol/L NaCl, and 1 mmol/L MgCl₂) was added to each well and incubated for 10 min. Developing solution (100 μL, containing 1 mol/L Tris base, 0.4 mol/L NaOFL 2 mmol/L TbCl₃, and 3 mmol/L EDTA) was pipetted into each well and mixed for 1 min. The fluorescence was then measured with a time-resolved fluorometer, the Cyberfluor 615 Immunoanalyzer (MDS Nordion, Kanata, ON, Canada). The calibration and data reduction were performed automatically, as described elsewhere (13).

Determination of the Sensitivity of the hK5 Immunoassay.

Recombinant hK5 was used to generate the calibration curve. hK5 calibrators were prepared by diluting the purified recombinant hK5 in the general diluent. These calibrators were then used to define the detection limit of the assay.

Determination of the Specificity of the hK5 Immuoassay.

Biological fluids and recombinant hK5 were used to determine the specificity of the developed immunoassay. These samples were first measured with the standard assay procedure described above. The mouse and rabbit anti-hK antisera were then successively replaced with sera from the same animals, obtained before immunization (preimmune sera). The samples were measured again, and the fluorescence counts were compared with the counts obtained by the standard assay. The cross-reactivities of other homologous proteins were investigated using purified recombinant hK1-hK15 (available in-house), all diluted in the general diluent, and measured using the same assay. All other kallikreins were calibrated by the bicinchoninic acid method using bovine serum albumin as calibrators.

Determination of Assay Precision, Linearity and Recovery.

To assess the precision of the assay, hK5 calibrators and various clinical samples (2 milks, 2 seminal plasmas and 3 amniotic fluids) were analyzed either within-run (10 replicates) or between-run (10 replicates over 2 weeks). To assess the linearity of the assay, 2 milks, 2 seminal plasmas, 2 amniotic fluids, 2 sera with elevated hK5 values, 1 skin extract and 1 ovarian cancer tissue extract were serially diluted with a 60 g/L bovine serum albumin and re-assayed. Recovery was assessed by adding recombinant hK5 to milk, ascites fluid from ovarian cancer patients and serum samples from men and women.

Preparation of Human Tissue Extracts and Biological Fluids

The following human tissues (adult and fetal) were used for screening: Esophagus, tonsil, skin, testis, kidney, salivary gland, breast, fallopian tube, adrenal, bone, brain, cerebellum, colon, endometrium, liver, lung, muscle, ovary, pancreas, pituitary, prostate, seminal vesicle, small intestine, spinal cord, spleen, stomach, thyroid, trachea and ureter. Human tissue extracts were prepared as follows: Frozen human tissues (0.2 g) were pulverized on dry ice to fine powders. Two mL of extraction buffer [50 mmol/L Tris-HCl buffer (pH 8.0) containing 150 mmol/L NaCl, 5 mmol/L EDTA, 1% NP-40 surfactant] were added to the tissue powders and the mixture was incubated on ice for 30 minutes with repeated shaking and vortex-mixing every 10 minutes. Mixtures were centrifuged at 14,000×g at 4° C. for 30 minutes. The supernatants representing the tissue extracts were collected and stored at −80° C. until use. The biological fluids (amniotic fluid, breast milk, cerebrospinal fluid, follicular fluid, serum and seminal plasma) were leftovers of samples submitted for routine biochemical testing and stored at −80° C. until use.

Preparation of cytosol fractions from normal tissues as well as from tissues of patients with benign ovarian disease or from tumor tissues of patients with primary ovarian cancer, at the Department of Obstetrics and Gynecology, Technical University of Munich, Munich, Germany was described previously by Schmalfeldt et al (14). Information on stage and grade were not available. Cytosol fractions were stored in liquid nitrogen until use. Ascites fluids were obtained from patients with ovarian cancer stage FIGO (International Federation of Obstetrics and Gynecology) II (n=9), III (n=18) and IV (n=4) by tapping the patients and collection of fluid into a sterile bag, after flushing the needle and bag with heparin. The ascites fluids were then centrifuged at 10,000×g for 30 minutes to sediment cells and any debris. The supernatants were harvested, aliquoted and stored at −80° C. until use.

Recovery

Recombinant hK5 was added to a general diluent (control), male and female sera and various biological fluids at different concentrations and the spiked samples were measured. Recoveries were then calculated after subtraction of the endogenous concentrations.

Fractionation of Biological Fluids with Size-Exclusion HPLC

To determine the molecular mass of the hK5 protein detected in the biological fluids and tissue extracts, various samples were fractionated with gel filtration chromatography, as described elsewhere (15). The fractions were collected and analyzed for hKS using the developed immunoassay. For comparison purposes, fractions for human kallikrein 6 (hK6) were also analyzed using a previously published assay (16).

Cancer Cell Lines and Hormonal Stimulation Experiments

The breast cancer cell lines MDA-MB-231, BT474, T-47D, ZR-75 and MCF-7, the ovarian cancer cell line HTB-75 (Caov-3) and the prostatic carcinoma cell lines LNCaP and PC-3 were purchased from the American Type Culture Collection (ATCC), Rockville, Md. The BG-1 ovarian cancer cell line was kindly provided by Dr. Henri Rochefort, Montpellier, France and the PC-3 cell line, stably transfected with androgen receptor [PC-3 (AR)₆] was kindly provided by Dr. Theodore Brown, Toronto, Canada. Cells were cultured in RPMI media (Gibco BRL. Gaithersburg, Md.) supplemented with glutamine (200 mmol/L) and fetal bovine serum (10%), in plastic flasks, to near confluency. The cells were then aliquoted into 24-well tissue culture plates and cultured to 50% confluency. Twenty-four hours before the experiments, the culture medium was changed into medium containing 10% charcoal-stripped fetal bovine serum. For stimulation experiments, various steroid hormones dissolved in 100% ethanol were added into the culture media, at a final concentration of 10⁻⁸ M and ethanol final concentration of 0.1%. Cells stimulated with ethanol were included as controls. The cells were grown for 7 days and the tissue culture supernatants were collected for hK5 quantification by fluorometric ELISA.

In Silico Analysis of KLK5 Expression

Analysis of KLK5 mRNA expression in a variety of normal and cancer cell libraries obtained from different tissues was performed using the database of the Cancer Genome Anatomy Project (CGAP) of the National Cancer Institute (NCI). Quantitative KLK5 transcript levels were assessed by “Virtual Northern” analysis of the Serial Analysis of Gene Expression database (SAGEmap). Comparison between normal and cancerous cDNA libraries was done using the Expressed Sequence Tag (EST) database of the CGAP through the “Digital Differential Display” (DDD) search engine, and the “SAGEmap” database through the “Xprofiler” search engine.

Results

Production and Purification of hK5 Recombinant Protein.

The cDNA encoding for the active form of hK5 was cloned into a P. pastoris yeast expression system. Expression in yeast produced a protein migrating around 35 kDa. The protein was purified by ion-exchange and reverse-phase chromatography essentially as described elsewhere for hK10 protein (17). The purified protein was then run on an SDS-PAGE gel, stained with Commassie blue and the band was excised from the gel and hydrolyzed by trypsin digestion. The tryptic digests were subjected to MALDI-TOF mass spectrometry and selected fragments were further sequenced by using tandem mass spectrometry, as described elsewhere (17). This analysis confirmed that the recombinant protein produced in yeast is human kallikrein 5. For example, the peptides LGHYSLSPVYESGQQMFQ [SEQ ID NO. 5] and DVRPINVSSHCPSAGTK [SEQ ID NO. 6] correspond to the amino acid sequences 118-135 and 168-184 of hK5 protein (GenBank Accession # AF135028). Since the predicted molecular weight of non-glycosylated human kallikrein 5 is approximately 32 kDA (5), it was assumed that the recombinant hK5 protein, like other kallikreins produced in Pichia, is glycosylated.

Recombinant hK5 protein was injected into mice and rabbits, as described above to generate polyclonal antibodies. High titers of specific antibodies were detected in serum of mice and rabbits after the third booster injection. These antibodies were used for the development of the hk5 immunofluorometric assay. A “sandwich-type” assay configuration was adopted in which the capture antibody was generated in mice and the detection antibody in rabbits. This assay configuration does not necessitate any prior antibody purification and was highly specific and sensitive.

Sensitivity, Specificity, Linearity and Precision of the hK5 Immunofluorometric Assay

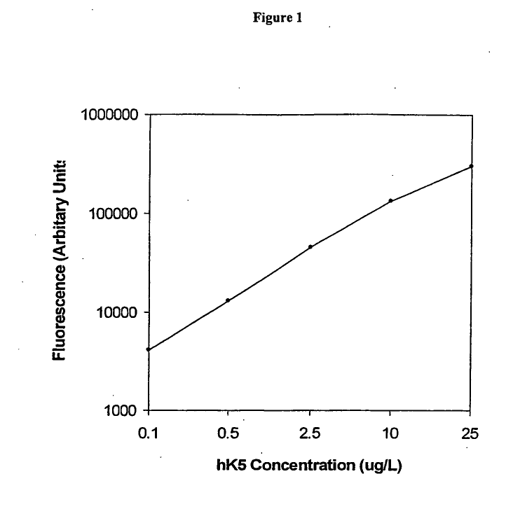

A typical calibration curve for the hK5 immunofluorometric assay is shown in FIG. 1. The detection limit, defined as the concentration of hK5 that can be distinguished from zero with 95% confidence, was 0.1 μg/L, and the dynamic range extends to 25 μg/L. It was further confirmed that the assay specifically measures hK5. When mouse and rabbit antisera were successively replaced with pre-immune mouse and rabbit sera, the fluorescence signals of standards or hK5-positive samples were reduced to nearly zero (data not shown). Since hK5 is a member of the human kallikiein family, it shares significant amino acid homology with other kallikreins (2). The hK5 protein shows 50-51% identity with hKS, 9, 11, 13 and 14, 45-49% identity with hK6, 7, 12 and 15 and 38-44% identify with hK1, 2, 3, 4 and 10. To demonstrate that there is no interference from these homologous proteins, the cross-reactivities of recombinant hK1, hK2, hK3, hK4, hK6, hK7, hK8, hK9, hK10, hK11, hK12, hK13, hK14 and hK15 were examined. All recombinant proteins produced no measurable readings, even at concentrations 1000-fold higher than hK5. These data demonstrate that this immunoassay can efficiently discriminate hK5 from other homologous proteins and that it measures hK5 with high specificity.

To assess the linearity of this assay, various clinical samples were diluted serially and hK5 was re-measured. These samples included milks, seminal plasmas and amniotic fluids. In all samples, a good dilution linearity was found with this assay, suggesting freedom from matrix effects. Within- and between-run precision was assessed with various hK5 calibrators and clinical samples over two weeks (n=10). In all cases, the CVs were between 3-8% within the dynamic range of the assay (0.1-25 μg/L).

hK5 Recovery from Biological Fluids.

The recovery of added recombinant hK5 was tested in 6% bovine serum albumin (BSA) (as a control), and in milk, ascites fluid and serum. Samples were incubated for 1 h at room temperature after spiking. Recoveries ranged from 90-100% in BSA, 80- 90% in milk, 60-75% in ascites fluid and 50-60% in male and female serum. About the same recovery was obtained when serum samples from males and females were spiked with native hK5 present from milk (data not shown). The recovery data in serum suggested that hK5 may be sequestered in serum by proteinase inhibitors, similarly to other kallikreins, including hK3 (PSA) and hK2 (19-22) (see also below).

Expression of hK5 in Human Tissues and Presence in Biological Fluids.

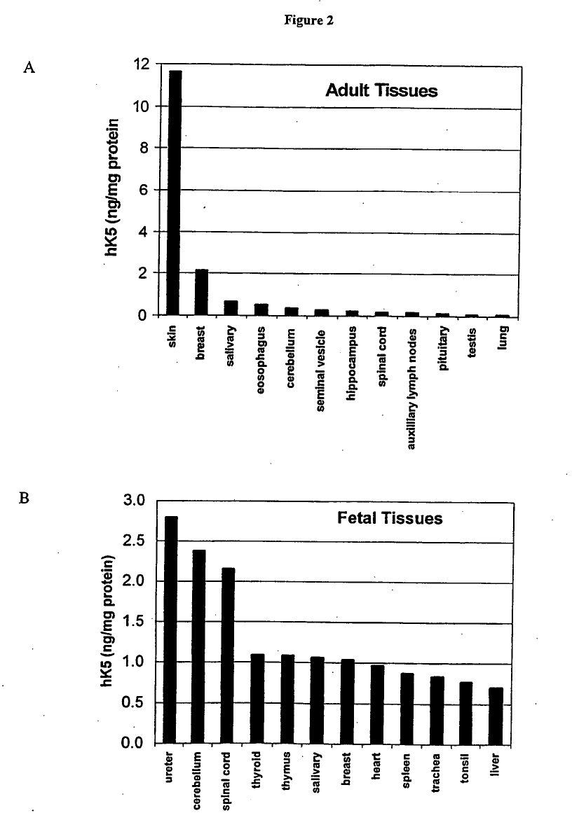

The expression of hK5 in human tissues was investigated by analyzing tissue extracts with the developed hK5 immunoassay. Tissues were collected at autopsy. The data for adult and fetal tissues are graphically shown in FIG. 2. Highest hK5 levels in adult tissues were seen in slkn, followed by breast, salivary gland, esophagus, cerebellum, seminal vesicle, hippocampus, spinal cord, axillary lymph node, pituitary, testis and lung. The following tissues were either weakly positive or negative: bone, kidney, colon, liver, muscle, pancreas, prostate, spleen, thyroid, stomach, small intestine, trachea, endometrium, fallopian tube, ovary, ureter, mesentery lymph node, uterus, tonsil, frontal cortex, medulla, midbrain, occipital cortex, pons and temporal lobe. In fetal tissues, highest levels were seen in ureter, cerebellum and spinal cord and lower levels in many other tissues (FIG. 2).

hK5 protein was also quantified in various biological fluids of human origin. Highest levels were seen in milk of lactating women. Among 6 samples, the range was 10-409 μg/L with a mean of 120 μg/L and a median of 61 μg/L. The other tested fluids (6 samples per fluid type) had much lower amounts (up to 0.17 μg/L for cerebrospinal fluid, up to 0.9 μg/L for amniotic fluid, up to 0.4 μg/L for seminal plasma and up to 0.35 μg/L for follicular fluid).

Ovarian Tissue Extracts.

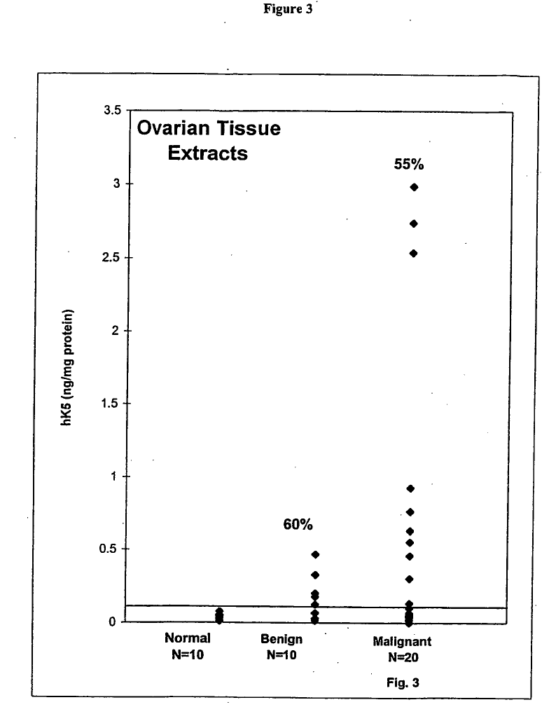

Cytosolic extracts from 10 normal ovarian tissues, 10 tissues from benign ovarian disease patients and 20 tissues from ovarian cancer were prepared. These tissues were obtained at the time of surgery and before initiation of any other treatment. hK5 protein was then quantified with the developed immunoassay. After correction for the total protein content of each extract, the results are graphically presented in FIG. 3. Clearly, hK5 protein expression is higher in about 55-60% of patients with either benign ovarian disease or ovarian cancer. However, the levels of hK5 protein in the ovarian cancer tissue extracts is significantly higher than levels in either normal or benign disease tissue extracts.

Ascites Fluid of Ovarian Cancer Patients.

hK5 was quantified in 31 ascites fluid samples obtained from patients with metastatic ovarian carcinoma (stages II, III and IV disease). All samples were positive for hK5, with values ranging from 1 μg/L to 300 μg/L, with a mean of 32 μg/L and a median of 6.8 μg/L. After correcting for total protein in these ascites samples, the hK5 concentration, expressed as μg of hK5 per gram of total protein was 0.094±0.068 (mean±SD), the range was 0.01-0.30 μg/g and the median was 0.07 μg/g. For these patients, information on age, serum CA125 and FIGO stage were also available. Statistical analysis has indicated that there were no significant correlations between ascites fluid hK5 concentration and either patient age, CA125 concentration or FIGO stage (p>0.05 by the Fisher's exact test, data not shown).

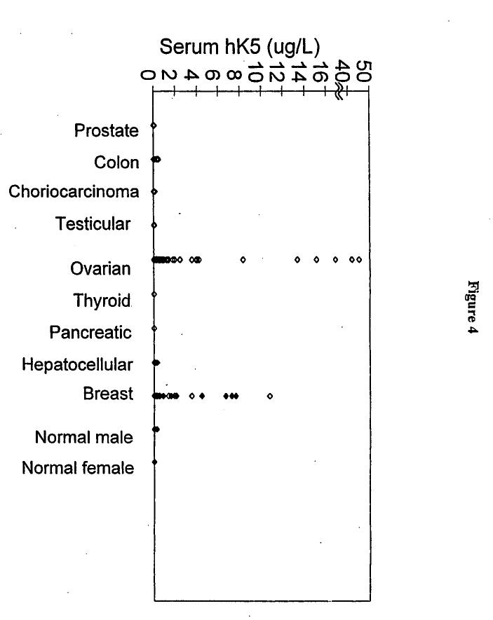

hK5 is a Serum Biomarker for Ovarian and Breast Cancer. A total of 263 serum samples were analyzed for hK5 by the developed ELISA assay. These samples were obtained from normal individuals (males, N=40 and females, N=40) and patients diagnosed with various malignancies including prostate (N=21), breast (N=39), ovarian (N=69), thyroid (N=10), colon (N=25), pancreatic (N=9) and hepatocellular carcinomas (N=10). The results are graphically presented in FIG. 4. A cut-off value of 0.1 μg/L was chosen, which represents the detection limit of the assay and the 100^{th} percentile of normals. None of the normals and only 4 (15%) of patients with colon cancer had hK5 concentrations slightly above the cut-off value (up to 0.4 μg/L). However, 67% of patients with ovarian cancer and 49% of patients with breast cancer had elevated hK5 levels in serum. In many of these patients, the serum levels were more than 20 times higher than the cut-off value (FIG. 4).

Fractionation of Biological Fluids with Size-Exclusion HPLC.

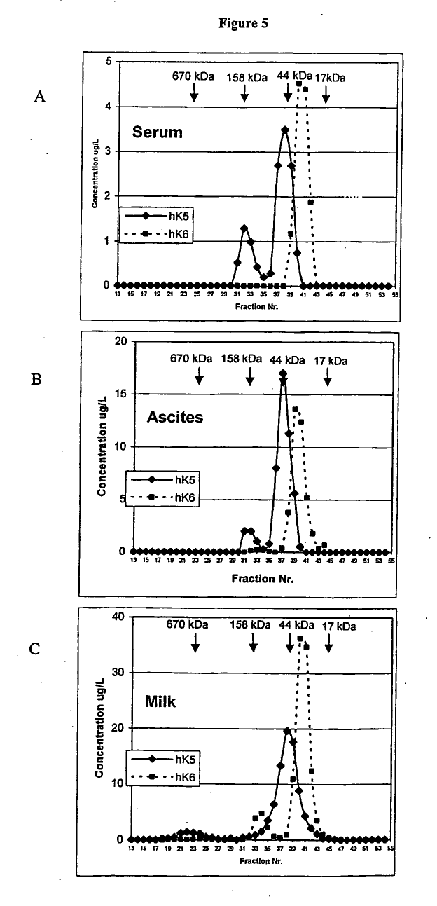

In order to determine the molecular mass of the protein detected in biological fluids, samples were fractionated on a gel filtration column. The presence of hK5 in the various fractions was then assessed with the developed immunoassay. For milk, when the hK5 concentration in fractions was plotted against the fraction number, a peak around fraction 38 (corresponding to a molecular mass of ˜50 kDa) was detected (FIG. 5). A small peak around the void volume of the column (˜700 kDa) could also be seen. In ascites fluid from ovarian cancer patients and in serum, another smaller peak corresponding to a molecular mass of ˜160-180 kDa was also detected. This likely represents hK5 bound to a proteinase inhibitor or another interacting protein. The higher than expected (30-35 kDa) molecular mass of hK5 in serum, ascites fluid and milk was verified by measuring another kallikrein, hK6, in the same samples (FIG. 5). It is possible that native hK5 is highly glycosylated or it may interact with the gel filtration column, leading to delayed retention.

Production of hK5 by Cell Lines and Hormonal Regulation.

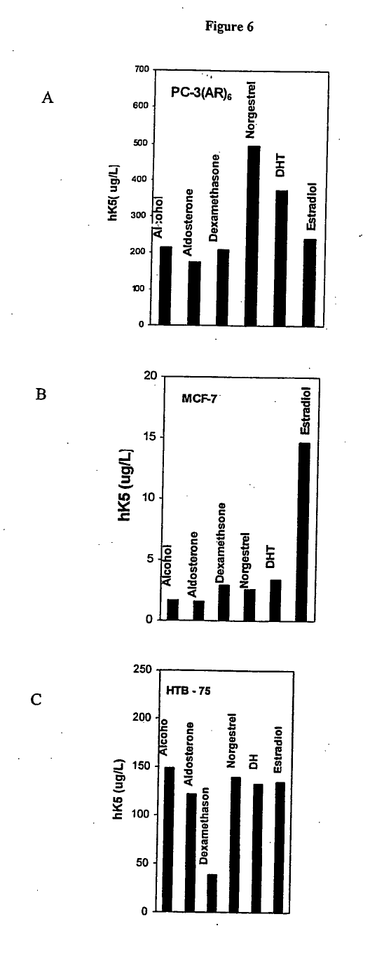

Several cancer cell lines were tested for constitutional hK5 expression and after steroid hormone stimulation. The cell lines PC-3 (AR)₆, HTB-75 (Caov-3) and MCF-7 produced relatively large amounts of hK5 while the cell lines MDA-MB-231, BG-1, T-47D, BT-474, PC-3, LNCaP and ZR-75 did not. Among the three cell lines producing hK5, PC-3 (AR)₆ secreted the highest levels (tissue culture supernatant concentration of approx. 1,300 μg/L, compared to 147 μg/L for HTB-75 and 1.5 μg/L for MCF-7 cells). These data refer to the supernatants collected from cells without any hormonal stimulation. Upon stimulation by steroid hormones, hK5 concentration increased by about 2-3-fold after norgestrel and dihydrotestosterone treatment in the PC-3 (AR)₆ cell line: A 3.5-fold reduction of hK5 concentration was also observed upon stimulation by dexamethasone in the HTB-75 (Caov-3) cell line and an 8-fold increase upon stimulation by estradiol in the cell line MCF-7 (FIG. 6).

In Silico Analysis of KLK5 Gene Expression

The expression pattern of KLK5 mRNA was analyzed using various CGAP databases and analysis tools. Virtual Northern analysis of a KLK5-unique SAGEtag (TCTCCTGGAC) [SEQ ID NO. 7] against the SAGEmap database indicated that while no expression was identified in normal ovarian libraries, KLK5 expression was detected in 5 ovarian cancer libraries from different sources. In addition, KLK5 was detected in 5 normal mammary gland libraries, consistent with the protein expression results of FIG. 2. The results of screening of the EST database for KLK5 ESTs are shown in Table 1. Out of 27 positive adult clones, 9 clones were from the same ovarian adenocarcinoma cell line, 6 from different other ovarian cancer libraries, 3 from squamous cell carcinoma of the skin, 2 from uterine cancer libraries and one from squamous cell carcinoma of the tongue. These data are suggestive of an association between KLK5 expression and certain malignancies (ovarian, skin, uterine, tongue). In addition, ESTs were isolated from testis, breast and lung libraries, consistent with the protein expression results.

Discussion

Many kallikreins have recently been shown to be differentially expressed in hormone-related malignancies (2, 3). Of particular interest is the link between kallikreins and ovarian cancer (2, 3, 8, 18, 23-29). hK6 (zyme/protease M) is a potential new serum biomarker for ovarian cancer (25). hK10 is also elevated in serum of ovarian cancer patients and has a role in diagnosis and prognosis (26). More recently, hK11 levels were shown to be elevated in serum of 70% of women with ovarian cancer (18). Herein evidence is provided showing that hK5 is a potential serum biomarker for ovarian cancer. The results are based on the finding of significant elevations of serum hK5 concentration in ovarian cancer patients (FIG. 4), and the identification of high levels of hK5 in ascites fluid from ovarian cancer patients and in ovarian cancer tissue extracts (FIG. 3). At the mRNA level, it has previously been shown that KLK5 is a marker of unfavorable prognosis in ovarian cancer (8).

KLK5 MnRNA can be used as a marker of unfavorable prognosis in breast cancer (9). Here, evidence is provided of serum hK5 protein elevation in a subset of patients with breast cancer (FIG. 4). It has been previously reported that another kallikrein, KLK10, is down-regulated in breast cancer (30-32) and that high protein levels in cytosols are associated with resistance to chemotherapy (33).

hK5 protein was previously purified from the human skin and shown to be activated by trypsin (6). However, hK5 was not previously detected in any other tissue or fluid due to the lack of specific reagents and sensitive analytical methods. Herein are described such reagents and methods. Using the highly sensitive and specific hK5 ELISA assay, hK5 was detected in various tissue extracts but predominantly in the skin, breast, salivary glands, esophagus and a few other tissues (FIG. 2). The tissue expression pattern found at the protein level correlates well with previous reports on mRNA expression, with highest levels found in skin and breast (2, 3, 5, 6). Relatively large amounts of hK5 were identified in milk, suggesting secretion of this protein by epithelial cells, similarly to another two kallikreins, hK6 and hK10, as shown by immunohistochemistry (34, 35). The concentration of hK5 in serum of healthy men and women appears to be very low, close to the detection limit of the assay (0.1 μg/L). The results of tissue expression and presence of hK5 in biological fluids are further supported by the Virtual Northern data and the summary of bioinformatic analysis of Table 1.

Many kallikreins circulate in biological fluids as complexes with proteinase inhibitors (19-22). The free, as well as the bound forms of these enzymes are useful biomarkers for the differential diagnosis of cancer (1, 22). In serum and ascites fluid, hK5 protein is present in two forms, one at a relatively lower molecular weight (around 50 kDa) and another one, around 150-180 kDa. These data suggest that hK5 likely interacts with proteinase inhibitors or other interacting proteins in these two fluids. Further evidence for such interaction is provided by the lower than expected recovery of added recombinant hK5 in biological fluids.

hK5 is expressed by prostate, breast and ovarian cancer cell lines and this expression is modulated by steroid hormones. As shown in FIG. 6, a significant upregulation of hK5 protein expression by estradiol was found in supernatants of MCF-7 breast cancer cells and up-regulation of hK5 protein expression by norgestrel and dihydrotestosterone was found in PC-3 (AR6) prostate cancer cell lines. Furthermore, a significant down-regulation of hK5 expression by dexamethasone in the HTP-75 cell line was observed (FIG. 6).

In addition to various experimental approaches, bioinformatic analysis of gene expression can now provide important information on the frequency of mRNA transcript abundance in various cDNA libraries. The data of Table 1 confirm the experimentally established expression of hK5 in ovarian, skin, testicular, breast and other tissues. Furthermore, and in accordance with the data of FIG. 3 and Table 1, it is clear that hK5 protein is significantly overexpressed in ovarian cancer, in comparison to normal tissues. Table 1 further suggests that hK5 measurements may be useful in cancers of the skin and endometrium.

In conclusion, this is the first evidence that hK5 concentration is elevated in serum of patients with ovarian and breast cancer. These data, combined with previous reports of higher serum and tissue concentrations of multiple kallikreins in ovarian cancer, suggest that this gene family may represent an enzymatic cascade pathway which is activated in ovarian and other cancers (36, 37).

The present invention is not to be limited in scope by the specific embodiments described herein, since such embodiments are intended as but single illustrations of one aspect of the invention and any functionally equivalent embodiments are within the scope of this invention. Indeed, various modifications of the invention in addition to those shown and described herein will become apparent to those skilled in the art from the foregoing description and accompanying drawings. Such modifications are intended to fall within the scope of the appended claims.

All publications, patents and patent applications referred to herein are incorporated by reference in their entirety to the same extent as if each individual publication, patent or patent application was specifically and individually indicated to be incorporated by reference in its entirety. All publications, patents and patent applications mentioned herein are incorporated herein by reference for the purpose of describing and disclosing the cell lines, vectors, methodologies etc. which are reported therein which might be used in connection with the invention. Nothing herein is to be construed as an admission that the invention is not entitled to antedate such disclosure by virtue of prior invention.

It must be noted that as used herein and in the appended claims, the singular forms “a”, “an”, and “the” include plural reference unless the context clearly dictates otherwise. Thus, for example, reference to “a cell” includes a plurality of such cells, reference to “peptide” is a reference to one or more peptide molecules and equivalents thereof known to those skilled in the art, and so forth.

Below full citations are set out for the references referred to in the specification.

TABLE 1
hK5 expression in normal and pathological conditions as estimated from EST analysis.

| | | Matching | | |
|---|---|---|---|---|
| Tissue | Library type | clones | Library ID | Source |
| Ovary | Adenocarcinoma cell line | 9 | NIH_MGC_9 | NIH¹ |
| Ovary | 5 pooled tumors | 1 | NCI_CGAP_OV23 | CGAP² |
| Ovary | Adenocarcinoma | 2 | NYH_MGC_66 | NIH |
| Ovary | Ovarian cancer | 2 | Soares ovary tumor NbHOT | LLNL³ |
| Ovary | Fibrotheoma | 1 | NCI_CGAP_ov18 | CGAP |
| Skin | Squamous cell carcinoma | 3 | NCI_CGAP_skn4 | CGAP |
| Endometrium | Adenocarcinoma cell line | 2 | NIH_MGC_44 | NIH |
| Tongue | Squamous cell carcinoma | 1 | NCI_CGAP_HN3 | CGAP |
| Testis | Normal testis | 1 | Soares_testis_NHT | LLNL |
| Stomach | Normal adult | 1 | ST0296 | LICR⁴ |
| Stomach | Normal adult | 2 | S17N258215 | CRIBB⁵ |
| Breast | Adult female | 1 | BT0590 | LICR |
| Larynx | Normal larynx | 1 | NCI_CGAP_Lar1 | CGAP |
| Lung | Fetal lung | 1 | Soares_fetal_lung_NbHL19W | LLNL |
| Heart | Fetal heart | 4 | Soares_fetal_heart_NbHH19W | LLNL |

¹National Institutes of Health, mammalian gene collection.
²Cancer Genome Anatomy Project.
³Lawrence Livermore National Laboratory.
⁴Ludwig Institute for Cancer Research.
⁵Korea Research Institute of Bioscience & Biotechnology.

### FULL CITATIONS FOR REFERENCES REFERRED TO IN THE SPECIFICATION

1. Rittenhouse, H. G., Finlay, J. A., Mikolajczyk, S. D., and Partin, A. W. Human Kallikrein 2 (hK2) and prostate-specific antigen (PSA): two closely related, but distinct, kallikreins in the prostate. Crit Rev Clin Lab Sci 35: 275-368, 1998.
2. Yousef, G. M. and Diamandis, E. P. The new human tissue kallik-rein gene family: structure, function, and association to disease. Endocr Rev 22: 184-204, 2001.
3. Yousef, G. M. and Diamandis, E. P. Human kallikreins: common structural features, sequence analysis and evolution. Current Genomics 4:147-165, 2000.
4. Diamandis, E. P., Yousef, G. M., Clements, J., Ashworth, L. K., Yoshida, S., Egelrud, T., Nelson, P. S., Shibsaka, S., Little, S., Lilja, H., Stenman, U. H., Rittenhouse, H. G., and Wain, H. New nomenclature for the human tissue kallikrein gene family. Clin Chem 46: 1855-8, 2000.
5. Yousef, G. M. and Diamandis, E. P. The new kallikrein-like gene, KLK-L2. Molecular characterization, mapping, tissue expression, and hormonal regulation. J Biol Chem 274: 37511-6, 1999.
6. Brattsand, M. and Egeirud, T. Purification, molecular cloning, and expression of a human stratum corneum trypsin-like serine protease with possible function in desquamation. J Biol Chem 274: 30033-40, 1999.
7. Yousef, G. M., Chang, A., Scorilas, A., and Diamandis, E. P. Genomic organization of the human kallikrein gene family on chromosome 19q13.3-q13.4. Biochem Biophys Res Commun 276: 125-33, 2000.
8. Kim, H., Scorilas, A., Katsaros, D., Yousef, G. M., Massobrio, M., Fracchioli, S., Piccinno, R., Gordini, G., and Diamandis, E. P. Human kallikrein gene 5 (KLK5) expression is an indicator of poor prognosis in ovarian cancer. Br J Cancer 84: 643-50, 2001.
9. Yousef, G. M., Scorilas, A., Kyriakopoulou, L. G., Rendl, L., Diamandis, M., Ponzone, R., Biglia, N., Giai, M., Roagna, R., Sismondi, P., and Diamandis, E. P. Human kallikrein gene 5 (KLK5) expression by quantitative PCR: An independent indicator of poor prognosis in breast cancer. Clin Chem 48:1241-1450, 2002.
10. Yousef, G. M., Obiezu, C., Jung, K., Stephan, C., Scorilas, A., and Diamandis, E. P. Differetial expression of kallikrein gene 5 (KLK5) in cancerous and normal testicular tissues. Urology 60:714-718, 2002.
11. Sambrook, J., Fritsch, E. F., and Maniatis, T. Molecular Cloning: A Laboratory Manual, Second Edition edition. NY: Cold Sping Harbor Laboratory, 1989.
12. Shevchenko, A., Wilm, M., and Mann, M. Peptide sequencing by mass spectrometry for homology searches and cloning of genes. J Protein Chem 16: 481-90., 1997.
13. Christopoulos, T. K. and Diamandis, E. P. Enzymatically amplified time-resolved fluorescence immunoassay with terbium chelates. Anal Chem 64: 342-6, 1992.
14. Schmalfeldt, B., Prechtel, D., Harting, K, Spathe, K, Rutke, S., Konik, E., Fridman, R., Berger, U., Schmitt, M., Kuhn, W., and Lengyel, E. Increased expression of matrix metalloproteinases (MMP)-2, MMP-9, and the urokinase-type plasminogen activator is associated with progression from benign to advanced ovarian cancer. Clin Cancer Res 7: 2396-404, 2001.
15. Yu, H. and Diamandis, E. P. Ultrasensitive time-resolved immunofluorometric assay of prostate-specific antigen in serum and preliminary clinical studies. Clin Chem 39: 2108-14, 1993.
16. Diamandis, E. P., Yousef, G. M., Soosaipillai, A. R., Grass, L., Porter, A., Little, S., and Sodiropoulou, G. Immunofluorometric assay of human kallikrein 6 (zy e/protease Mneurosin) and preliminary clinical applications. Clin Biochem 33: 369-75, 2000.
17. Luo, L. Y., Grass, L., Howarth, D. J., Thibault, P., Ong, H., and Diamandis, E. P. lmsunofluorometric assay of human kallikrein 10 and its identification in biological fluids and tissues. Clin Chem 47: 237-46, 2001.
18. Diamandis, E. P., Okui, A., Mitsui, S., Luo, L. Y., Soosaipillai, A., Grass, L., Nakamura, T., Howarth, D. J., and Yamaguchi, N. Human kallikrein 11: A new biomarker of prostate and ovarian carcinoma. Cancer Res 67: 295-300, 2002.
19. Stenman, U. H., Leinonen, J., Alfthan, H., Rannikko, S., Tuhkanen, K, and Alfthan, O. A complex between prostate-specific antigen and alpha 1-antichymotrypsin is the major form of prostate-specific antigen in serum of patients with prostatic cancer: assay of the complex improves clinical sensitivity for cancer. Cancer Res 51: 222-6, 1991.
20. Lilja, H., Christensson, A., Dahlen, U., Matikainen, M. T., Nilsson, O., Pettersson, K, and Lovgren, T. Prostate-specific antigen in serum occurs predominantly in complex with alpha 1-antichymotrypsin. Clin Chem 37: 1618-25, 1991.
21. Saedi, M. S., Zhu, Z., Marker, K, Liu, R. S., Carpenter, P. M., Rittenhouse, H., and Mikolajczyk, S. D. Human kallikrein 2 (hK2), but not prostate-specific antigen (PSA), rapidly complexes with protease inhibitor 6 (PI-6) released from prostate carcinoma cells. Int J Cancer 94: 558-63, 2001.
22. Stephan, C., Jung, K, Lein, M., Sinha, P., Schnorr, D., and Loening, S. A. Molecular forms of prostate-specific antigen and human kallikrein 2 as promising tools for early diagnosis of prostate cancer. Cancer Epidemiol Biomarkers Prev 9: 1133-47, 2000.
23. Yousef, G. M. and Diamandis, B. P. Expanded human tissue kallikrein family- novel potential cancer biomarkers. Tumor Biol. Tumor Biol 23:185-192, 2002.
24. Diamandis, E. P. and Yousef, G. M. Human tissue kallikrein gene family: a rich source of novel disease biomarkers. Expert Rev. Mol. Diagn 1: 182-190, 2001.
25. Diamandis, E. P., Yousef, G. M., Soosaipillai, A. R., and Bunting, P. Human kallikrein 6 (zyme/protease M/neurosin): a new serum biomarker of ovarian carcinoma. Clin Biochem 33: 579-583, 2000.
26. Luo, L. Y., Katsaros, D., Scorilas, A., Fracchioli, S., Piccinno, R, Rigault de la Longrais, I. A., Howarth, D. J., and Diamandis, E. P. Prognostic value of human kallikrein 10 expression in epithelial ovarian carcinoma Clin Cancer Res 7: 2372-9, 2001.
27. Dong, Y., Kaushal, A., Bui, L., Chu, S., Fuller, P. J., Nicklin, J., Samaratunga, H., and Clements, J. A. Human kallikrein 4 (KLK4) is highly expressed in serous ovarian carcinomas. Clin Cancer Res 7: 2363-71, 2001.
28. Underwood, L. J., Tanimoto, H., Wang, Y., Shigemasa, K, Parnley, T. H., and O'Brien, T. J. Cloning of tumor-associated differentially expressed gene-14, a novel serine protease overexpressed by ovarian carcinoma. Cancer Res 59: 4435-9, 1999.
29. Tanimoto, H., Underwood, L. J., Shigemasa, K., Yan Yan, M. S., Clarke, J., Parmley, T. H., and O'Brien, T. J. The stratum corneum chymotryptic enzyme that mediates shedding and desquamation of skin cells is highly overexpressed in ovarian tumor cells. Cancer 86: 2074-82, 1999.
30. Liu, X. L., Wazer, D. B., Watanabe, K., and Band, V. Identification of a novel serine protease-like gene, the expression of which is down-regulated during breast cancer progression. Cancer Res 56: 3371-9, 1996.
31. Goyal, J., Smith, K M, Cowan, J. M., Wazer, D. E., Lee, S. W., and Band, V. The role for NES1 serine protease as a novel tumor suppressor. Cancer Res 58: 4782-6, 1998.
32. Dhar, S., Bhargava, R, Yunes, M., Li, B., Goyal, J., Naber, S. P., Wazer, D. E., and Band, V. Analysis of normal epithelial cell specific-1 (NES1)/Kallikrein 10 mRNA expression by in situ hybridization, a novel marker for breast cancer. Clin Cancer Res 7: 3393-8, 2001.
33. Luo, L. Y.,. Diamandis, E. P., Look, M. P., Soosaipillai, A. P., and Foekens, J. A. Higher expression of human kallikrein 10 in breast cancer tissue predicts tamoxifen resistance. Br J Cancer 86: 1790-6, 2002.
34. Petraki, C. D., Karavana, V. N., Skoufogiannis, P. T., Little, S. P., Howarth, D. J., Yousef, G. M., and Diamandis, E. P. The spectrum of human kallikrein 6 (zyme/protease Mneurosin) expression in human tissues as assessed by immunohistochemistry. J Histochem Cytochem 49: 1431-41, 2001.
35. Petaaki, C. D., Karavana, V. N., Luo, L.-Y., and Diamandis, E. P. Human kalliklrein 10 expression in normal tissues by immmunohistochemistry. J. Histochem. Cytochem. 50:1247-61, 2002.
36. Yousef, G. M. and Diamandis, E. P. Kallikreins, steroid hormones and ovarian cancer: is there a link? Minerva Endocrinol 27: 157-66, 2002.
37. Yousef, G. M. and Diamandis, E. P. Human tissue kallikreins: a new enzymatic cascade pathway? Biol Chem, 383: 1045-1057, 2002.

1-2. (canceled)

3. A method as claimed in claim 5 wherein the kallikrein 5 is detected using antibodies specifically reactive with kallikrein 5 or a part thereof.

4. (canceled)

5. A method for detecting or screening a subject for breast or ovarian cancer comprising (a) obtaining a biological sample from a subject; (b) detecting the amount of kallikrein 5 in said sample; and (c) comparing said amount of kallikrein 5 detected to a predetermined standard, where detection of a level of kallikrein 5 greater than that of a standard is indicative of breast or ovarian cancer.

6. A method of claim 5 wherein the level of kallikrein 5 is 2×, 5×, 10×, 15×, or 20× the levels of the standard.

7-8. (canceled)

9. A method for monitoring the progression of breast or ovarian cancer in a subject, the method comprising:
(a) detecting in a sample from the subject at a first time point, kallikrein 5;
(b) repeating step (a) at a subsequent point in time; and
(c) comparing levels detected in steps (a) and (b), and thereby monitoring the progression of breast or ovarian cancer.

10. A method of claim 5 for screening a subject for breast or ovarian cancer comprising
(a) incubating a biological sample from the subject with a first antibody specific for kallikrein 5 which is directly or indirectly labeled with a detectable substance, and a second antibody specific for kallikrein 5 which is immobilized;
(b) detecting the detectable substance thereby quantitating kallikrein 5 in the biological sample; and
(c) comparing the quantitated kallikrein 5 with levels for a predetermined standard.

11. A method of claim 5 wherein the biological sample is serum.

12. A method of claim 5 which further comprises detecting one or more of human stratum corneum chymotryptic enzyme (HSCCE), haptoglobin alpha, osteopontin, kallikrein 2, kallikrein 3, kallikrein 4, kallikrein 6, kallikrein 7, kallikrein 8, kallikrein 9, kallikrein 10, kallikrein 11, kallikrein 13, kallikrein 14, kallikrein 15, CA125, CA15-3, CA19-9, OVX1, lysophosphatidic acid (LPA) or carcinoembryonic antigen (CEA).

13. A method for assessing the potential efficacy of a test agent for inhibiting breast or ovarian cancer in a subject, the method comprising comparing: (a) levels of kallikrein 5 in a first sample obtained from a subject and exposed to the test agent; and (b) levels of kallikrein 5 in a second sample obtained from the subject, wherein the sample is not exposed to the test agent, wherein a significant difference in the levels of expression of kallikrein 5 in the first sample, relative to the second sample, is an indication that the test agent is potentially efficacious for inhibiting breast or ovarian cancer in the subject.

14. A method of claim 13 wherein the first and second sample comprises portions of a single sample obtained from the subject.

15. A method of claim 13 wherein the first and second sample comprises portions of pooled samples obtained from the subject.

16-25. (canceled)

26. A kit for assessing whether a subject is afflicted with breast or ovarian cancer, the kit comprising reagents that specifically bind with kallikrein 5.

27. (canceled)

28. A kit as claimed in claim 26 wherein the reagents are antibodies that specifically bind with protein or protein fragments corresponding to kallikrein 5.

29. A method of claim 5 wherein the standard comprises normal levels of kallikrein 5 in samples of the same type obtained from control subjects not afflicted with breast or ovarian cancer.

30. A method of claim 29 wherein the level of kallikrein 5 in the sample is 2×, 5×, 10×, 15×, or 20× the normal levels.

31. A method of claim 9 wherein the biological sample is serum.

32. A method of claim 10 wherein the biological sample is serum.

33. A method of claim 13 where the significant difference is a decrease in the levels of expression of kallikrein 5.


SEQUENCE LISTING (7 sequences)

SEQ ID NO: 1 - protein, 293 aa, Homo sapiens
  ref <308>: DATABASE ACCESSION NUMBER: GenBank: AAD26429
  ref <309>: DATABASE ENTRY DATE: 2000-06-27
  ref <313>: RELEVANT RESIDUES: (1)..(293)
MATARPPWMWVLCALITALLLGVTEHVLANNDVSCDHPSNTVPSGSNQDLGAGAGEDARSDDSSSRIINGSDCDMHTQPWQAALLLRPNQLYCGAVLVHPQWLLTAAHCRKKVFRVRLGHYSLSPVYESGQQMFQGVKSIPHPGYSHPGHSNDLMLIKLNRRIRPTKDVRPINVSSHCPSAGTKCLVSGWGTTKSPQVHFPKVLQCLNISVLSQKRCEDAYPRQIDDTMFCAGDKAGRDSCQGDSGGPVVCNGSLQGLVSWGDYPCARPNRPGVYTNLCKFTKWIQETIQANS

SEQ ID NO: 2 - DNA, 11570 bp, Homo sapiens
  ref <308>: DATABASE ACCESSION NUMBER: GenBank: AF135028
  ref <309>: DATABASE ENTRY DATE: 2000-06-27
  ref <313>: RELEVANT RESIDUES: (1)..(11570)
gggcccagagtgaaggcaagagaaggagttgagagctccctctgcaaagtggcttgagtctcccctgcctaaaatgcagggagagggaggcagaaagacagggaagaggaaggggtggggaagaaagagagagagagagagagacagaataacacaactacagaaacacagagagaacacacagagagcctgggacacagggacacacagagtcagagagaaaagagaagatagagaaagacacaaatggagacacagaggtgtaaagaaagagagattaacagagtcccagatacacgcaaaggggcagaagcacagttttcagggtggtgtctatgatcatcttcttttttttttttttttttttttttttttgagacggagtctcgctctgtcgcccaggctggagtgcagtggcgggatctcggctcactgcaagctccgcctcccgggttcacgccattctcctgcctcagcctcccaagtagctgggactacaggcgcccgccactacgcccggctaatttttttgtatttttagtagagacggggtttcaccgttttagccgggatggcctcgatctcctgacctcgtgatccgcccgcctcggcctcccaaagtgctgggattacaggcgtgagccaccgcgcccggccatgatcatcttcttgactatgctgatgtgacaagtacctaaagccatcagactctaccctttaaatatgcagtttgggccaggcaccgtggctcatgcctgtaattccagcactttgggaggcagaggtgggtgaatcacttgaggccaggagtttgagaccagcctggccaacatggtgaaactctgtctttactaaaaaaaaaaaaaaaaaaaaaaaaatcagccgggtgtcgtggggcacacctgtaatcccagctatgctggaggctgaggcacgagagtcacttgaaccctggaggcggaggttgcagtgggccgagatcacatcaccgccctccagcctgggcgacagagcaagactctgtctcaaataaataaataaacaaacgaacaagcagtttgttgtaccttagttatatctaaaaaaaaaatgctgtcaacaaatagagcagaagtgaaataaaggaaaataaatgggccaagaactctaaggtatatttgacaaatcattcagaacctttaaaaaagaaagaatcacagaggcatagaaagacagggaggaacagggagacagaaacacctgtggcccaaggagaacaaaacaaggctcctaagacagacaggaggagagagagagagagtgagtgagagacagacagagaaaaagacagagagagagagacagagacagagagacagagaggcgagagggatagaaagagagagaggggtggagagagacacgagatattgagagagactcagaaagatagccgagggagaaccacagagagatggaagaagactctgagaaaaaaccagagacaaagatggaaagaggagtatcgagggtgaacagacagtggtggaatgagcaaaatgcagagaagaaagcaagcaatccaggcgccaagaatagtgacccagagttggtgagaagccagatccttaaggctgggggaggcagggaaggggctggcctggcttccggagacccctccccattctccgggccagggaggtagggagtgacattccggactgggtggggggtgctctgggggtggagatagggggagcaggaggagctattgctaaggcccgataggcacctcattgcccgggaatgtgccccagggagcagtgggtggttataactcaggcccggtgcccagagcccaggaggaggcagtggccaggaaggcacaggcctgagaagtctgcggctgagctgggagcaaatcccccaccccctacctgggggacagggcaagtgagacctggtgagggtggctcagcaggcagggaaggagaggtgtctgtgcgtcctgcacccacatctttctctgtcccctccttgccctgtctggaggctgctagactcctatcttctgaattctatagtgcctgggtctcagcgcagtgccgatggtggcccgtccttgtggttcctctctacctggggaaataaggtaggggagggaggggaagtgggttaagggctccccggatcgcctgggcctcccaaccctctgacattccccatccaggtgcagcggccatggctacagcaagacccccctggatgtgggtgctctgtgctctgatcacagccttgcttctgggggtcacaggtaaccagaactctggggtgggagggttgtgggattgggaggactgtctctgcggcactagagcgcctgtcccctggggaactgtgtgagcctgggcatgactccgggaccgggtgaatgtgagtctctgtctgtacttgtggttgtgcgatcgtatgtggccctgtgactgccacggtgtgtgtcggggagggggatgccttttcccatatcaggtgactgtgcggcaggtggcactgaccctttgaggctgtgtgtgtggttttgtgattgtgtgtgcatttaagattgtgtgtggctccacagctgtgtgggtgaatgcatgtagcactgggggtgttcactgtgtgtttggctgtgtgtggtgacttggcattgtatatgactgcaggtatctgcagttcctgtccctgaggtcccgggattgcgtgcaacaaaagtggtcatcaccatggaaagctgtgactgtgtgctgcttgcaggcgattatgtgattgtggctgagtgtgacgttatggatgcccgtatttgtgaccgtgtgactacctgaagctctgtgtaggggtgactgtatgtgactgtgtgtgtctgtgtgaggccgtgtaaatgctactgtatgtgtgatggtgcagctgtgtgtctggagtttctgtctctgcctggagggatagagggtgcaggggtagctatctctgggagatgggtgccaggtgactgacttgcagtgtgtgcctgtgtgcagaagagtatgtggcagtctgaacatctgtgcacacacggcatctgtgcgtggcactgagacactgtggatgagggtgtgcgatcccgctaggctgcccgggagcgtgtgtacctggagacagagctgtatgttagctgcacctgtggaggcaacatgggcgtgtctgcagaactgcgtgcgtgcttggctgttactgctgttgtgcgcgtggttcttggggtgagttcgtgaatgatggtggtgccagggccatcagcaagggtaagaaccaggccgggcgcggtggctcacgcctgtaatcccagccctttgggaggccgaggcaggcggatcacctgaggtcgggagatcgaggccagcctgaccaacatggagaaccccgtctctactaaaaatacaaaaaattagctggtgtggtggcgcgtgcctgtaatcccagctactcgggagactggggcagaaaaatcgcttgaacccgggaggtggaggttgcggtgagccgagatcgcgccattgcactccagcctgggcaacaagagcgaaactccgtctcgaaagaaaaaaagaaaaaaaaaagggtaagaaccagtgaatgggcacgggaggactgatgatggagtggggcatgcatgtagtctgtaggtctgtgtgtgagaggaggagattgacaggattgagaaggcatgttttcatctgagaattcagaaacctaggcctgctcttcccctccatgtggccccctaagctgagcccttctttcctggtcctgctttcggaaccctagctccgcccatgagctctgaccccacctcctttcctcaaccacgcccctaggccagactctagtggaccccgcctaaggccacacccctttgggccaggctccaccccctattctgtgggtaccttctagaacccccttcaaagtcagagcttttttttttttttttttggagacagtcttgctctctctcccaggctggagtgcagtggcgtgatctcggctcactgcaacctctgcctcccaggttcaagtgattctcgtgcctccacctcctgagtagctgggattacaggtgcgcgccaccacgcctggctaatttttgtgtctttagtagagacagggtttcaccttgttggccaggctggtctcaaactcccaacctcaggtgatccgcccacctcggcctcccagagtgctggggttacaggcgtgagccaccgcccccagcccaaagtcagagctctttataggagactctaacatgtaaccctgaccctggccctaactaagtcaattccaaaccccttcctgcctccagccctgaccccactcactgaggcctgaccccacttcttgagaccagttccatccctaaagccctggtctccctcccatccccaggctccagcccccacagctttggcactacccctgagcttgtccaggaatcctgtacccaattttaccctcacatgtagttctagccaattccaggaatctgtgaggtccagttagagtccagtaaccctacctgagcctgggctctgtccttgagcttgagcctgggcttgagaggtgccactcttattctccaggccctgcccctgccccctcagcatgtcagacacccaccctctagctggtctggcctcttgagtctgaaacccacccccagcccaagccccgcctctgagccccgcccaacccattttccgttcccagagcatgttctcgccaacaatgatgtttcctgtgaccacccctctaacaccgtgccctctgggagcaaccaggacctgggagctggggccggggaagacgcccggtcggatgacagcagcagccgcatcatcaatggatccgactgcgatatgcacacccagccgtggcaggccgcgctgttgctaaggcccaaccagctctactgcggggcggtgttggtgcatccacagtggctgctcacggccgcccactgcaggaagaagtgagtgggagttccaagaggagggttggtggggacggggaagtgggggtgggggtggggaagtgggggtgggggtgtcatggaggtgagggctggtggggacggggaagtggggttgggggtgtcatggaaggtgagggttggtggggatgggttggggatgtgggagcaggaggaggtcgagttggggataggactaaggatggagttttgcgggggagcaaggtgggaggatgaggttggagaggggagagtgttgtggtagggaatgggaaggagccaaggatgggttggatttggggttaggagcatatatttgttgaatggtttgggatggaggtggaattgggattggctttagaattgggggtgggtgaaaatcgggctggggtggaaatgaagatagcatggagatagggttgagattgggagcagatatagaatgaaggatggggattggagttttgggtggggttggagatggttggatttgggcttgagaatgcatatggtgatggcttctgggtagggaaagaattagggttgggaatgggatgggtttggaattgtgactgggatggggacaggcatgggattggagaccaagagggagttgaggatggtttggggaccgggggtggggatgggggtggggctggggctgggtgtggggttgggattggcgttggacgtggagatagagatcagggttggtggtgacctgccccatcttcctcagagttttcagagtccgtctcggccactactccctgtcaccagtttatgaatctgggcagcagatgttccagggggtcaaatccatcccccaccctggctactcccaccctggccactctaacgacctcatgctcatcaaactgaacagaagaattcgtcccactaaagatgtcagacccatcaacgtctcctctcattgtccctctgctgggacaaagtgcttggtgtctggctgggggacaaccaagagcccccaaggtgagtgtccaggttcttcttgataccgacccatctctgccgccttccatctttctccacttctcattgtgttcctgtttgacagtgcacttccctaaggtcctccagtgcttgaatatcagcgtgctaagtcagaaaaggtgcgaggatgcttacccgagacagatagatgacaccatgttctgcgccggtgacaaagcaggtagagactcctgccaggtgaggacacctctctttattcagcagatacacactgagtgccaactcggtaacatggagcgttgccaaattctgagaatccagcaattgccaagacagtcaggacccctgttctcacagagctcataccctagagtagtggtgtttagtagaaataatgctgagctgcttatgtcatttccagttttttagtagccacattaaaacaggtaaaaaaggctgggcgcagtggctcacacctgtaatcccagcactttgggaggctgaggcaggcagatcacctttggtcaggagtttgagactagcctggccaacatggcgaaactctgtctctaaaaaaaaatacaaaaattagcctggcatggtggcgggcgcctgtaatctcagctgctcaggaggccgagacacaagaatcacttaaacccaggaggtggaggttgcagtgagctgagatcgtgccactcactccaacctgggagacagagtgacacttttgtctcaaaaagaaaaaaaaaaacaagtaaaaaagaaacaggtgaagttaactttaataacccaatgtatcccaaatacaatcatttcaaagtgtaattaatataaaacaattatgaatgagatactttacattcttttcttgttttcatattaagtctttgaaagtgagtatatatgttatgctgacagcacatctcaatttggactagctacatttcaggtgctcagtagccacatgtggctagcagttactgtattggatggcacggatctagagggaaagatcagggctgttttgtatggttgggcaggttgtgcactgcataaagataccatatctaataggggcactccgtgttacagatgtcagttttggcagttttcaggcgtgtggtagttaagtgtcttgtttcaacaaaatctgtaatatgacagttttctagcaagtgctggtaaaatatcttgaggaaggaaaagagaaatctggtaggtatttttacaagagaatatttaatacaggggattaattgcaaagctgctggaagggctggaggaacaaagttaaaaaataaaaaactctgtggtcaagaatctgcataaatagggcaatttcagagagtggtaaaggttaaccccaaaataaaacatggttttaggatagtaaacaataagggccaatattcaaaaaggtggtcaggggagcctccttggagaggtggcatttgagcagagaatggatgacacaaagaagctaaactcgtgaagtttaaggggaaagaaaaggcacgtgcaaaggccctgaggcagtaaggaatttggctgattcaaagaagaagaggaaaccaatgcaactggagaacaaaagtgggggcaacagtagaaagtgacgctggaggtgtaggcaggggcgaatgctctgcaagtatttcttggtcaccaacacagagcttccctatgttctaatggaagctgtatctgttgaggaagacagaatttaaaatcaaactgttacatcaaccagcacccttctctgtattcaggctcccaagggatctagaaggacgtaagttaacaagctctcattagcagggtgtgtgtttcaacagtagttaggaagctggggattcaggagtactccagtcccatggctatgaaaagctccccccaaattgtacaaacctgacaaatgcaacacctccccagctctccccatttcttctctgtgccctgggtgtgggggggtgggttgcgagggggaaaacttttaacagaagaaagcacatctcggccgggcgtggtggctcacacctgtaatcccaacactttgggaggccgaggcgggtggatcactaggtcaggagatggagaccatcctggctgacacggtgaaaccctgtctctactaaaaacacaaaaaattagccgggcgtggtggcaggcgcctgtagtcccagctactcgggaggctgaggcaggagaatggcctgaacccgggaggcggaacttgcagtgagccgaggttgcaccactgcactccagcctgggcaacacagtgagactccgtctcaaaaaaaaaaaaagaaaagaaaagaaatcacatctcattcaagtggtggcatttaaaactatttagcctttctgtaggcaaggttagtatcttgtttttccagacctcaaggtgtttttttgtttgttttttcataccggtgtgtggtctgggtgtggccactaaaagctacaagcaagaaataataacaactacaacaatactaataccaatagtataaaaataatagcatctggctaattgctggacactgttttaagtggtttgcatgcctcagctcattaactcatttacctgttattattggccctattttacaaacaaggagccaaggctcagagcagttaactaacagcctctcaaaagaaactctgcagagatattaaatttaaaaaataatgagagaaattaaaccacaagaaagttgaaatttagaggtacaggcagctaagcttgtttgctttgaaacagtgtctgctactgggaaaaaggcaagtcttggctttcctaataattgataccaggactctgtaattcatattttgcatgcatgtaagtaagaaatgaagccgggtgcaatggcacatgccagtaatcccagcactctgggagactgaagtgggaagatcacttgagctcaggagttcaagaccagcctgggcaactaaaaattaaaaaaataaaaatactaattgtttttattttagtagattttattcataccacttacatcattattgtagtatgtacatatttatttcttttcttttcttttcttttcttttttgagacggagtctcgctctgtcacccaggctggagtgcaatggcaccatatcagctcactgcagcatgcgcctcctgggttcaagcatttcttccacctcagcctcccaagtagctgggataacaggcacccaccaccatgcctggctatttttttttttccgtagagatggggttccaccatgttggccaggctggtcttgaactcctgacctccagtgatctgcctgcctcggcctcccaaattgctggtattacaggtgtgagccaccgtgcccaggtgggagatagacatttctctctacctcaaacagaggtccactcaagctacttttcattttcttcataaatattagccgagtggctattttgcaccaggaatggttccaggtgctgtggatatggcatcaggcaaaacagaccaaaaacttcctgccgcgtggacctcatgttccccaagtggaagacaggcaataaagagatagataaatatgtagtaaattaaaaaaaaaaaaaattagccgggtgtggtggcttgcacctgtagttccagctacttgggaggctgaggtgggagaattgcttgagcccaaacgtttgaggctgcggtaagccatgactgcactgctgcactccagacagcagcctgggtgacaaagcaagacgtttttgtcagaaagaaaaaaaaaagagacgaagggaggaaggagagagaaaggaaggaaggaaggagaaagaaaggaaggaaggagaaagaaaggaaggaaggaaggagaaagaaaggaagaaagagaaagaaagaaaaagaaagaaagaaagaagaaagaaaagagagaggaaggaaggaaagaaggaaaagagggaaaaaaatgactgttgaagagcagtgagtattattataggagggtaattatagggaggtatggggaattgaagacaggaaacacaaattagtccaagcgaatggatttctattgggagtgattctgcccctagaagacactggcaataccaggagacatttttggttgtcacaactatatggaggggcattactggcaactaatggatagatgccaagtgtgctgttcaacatgctatgatgcacacggcaggcctccacaacaaaccattatccagcttcagatgcccacagtgcccagatcgaggaaccctcatccaggggctgagaaccgtatttttgcagaagggaggtataaggatgggttggtggagaatggggaaggaaggtgtgtgtccagtaagagaaataaggcctgcacaggctggaggggagagtgagagagaaagggaggcggagagatacacgatgagggagacaggctggaacagaaagtagagacgaagattcgagatgtggagaggaagggtcacagacccccccgaaatgatgtgtggacaacaggaatctggaagaggaagatggagtggagagtgacaaatggggtctaaaggttgaacttggaggccaggcatggtggctcacgcctgtaatcccaacactttggaggctgaggtgggcgaatcacttgaggccaggagttcgagaccagcctggccaacatggtgaaaccccgtctctacaaaaaaaatacaaaaaattagccgggtgtggtgatggacacctgtagtcacagctacttgggaggctgaggcaggagaattgcttgaacccgggagatggaggctgcagtgagctgaggtcaggccactgcgctccaacctgggcaacagagtaagactccatctcaaaaaaaaaaaagctggatttggagtgaaatattaataacattctccctctctctccttttgcctgtgtctccatctctgtctttttctgcatttcttcatctctgtactttccatctctgtgtgtctgttcccatctgcttctccatctatgggcatctctgggtctctcatgtctccttctgcccactttgccacatctctgcctctctcatgcccccctttctctcctgcagggtgattctggggggcctgtggtctgcaatggctccctgcagggactcgtgtcctggggagattacccttgtgcccggcccaacagaccgggtgtctacacgaacctctgcaagttcaccaagtggatccaggaaaccatccaggccaactcctgagtcatcccaggactcagcacaccggcatccccacctgctgcagggacagccctgacactcctttcagaccctcattccttcccagagatgttgagaatgttcatctctccagcccctgaccccatgtctcctggactcagggtctgcttcccccacattgggctgaccgtgtctctctagttgaaccctgggaacaatttccaaaactgtccagggcgggggttgcgtctcaatctccctggggcactttcatcctcaagctcagggcccatcccttctctgcagctctgacccaaatttagtcccagaaataaactgagaag

SEQ ID NO: 3 - DNA, 29 bp, Artificial
  Primer
agcagccgatcgatcaatggatccgactg

SEQ ID NO: 4 - DNA, 29 bp, artificial
  Primer
ctgagtcctgggatgtctagagagttggc

SEQ ID NO: 5 - protein, 18 aa, homo sapiens
LGHYSLSPVYESGQQMFQ

SEQ ID NO: 6 - protein, 17 aa, homo sapiens
DVRPINVSSHCPSAGTK

SEQ ID NO: 7 - DNA, 10 bp, artificial
  SAGE tag
tctcctggac